(19) United States
(12) Patent Application Publication
(10) Pub. No.: US 2024/0309044 A1
(43) Pub. Date: Sep. 19, 2024

(54) ANION EXCHANGE CHROMATOGRAPHY PROCESSES USING A PRIMARY AMINE LIGAND

(71) Applicant: AMGEN INC., Thousand Oaks, CA (US)

(72) Inventors: Joseph Edward Basconi, Cambridge, MA (US); Andrew John Maloney, West Roxbury, MA (US); Nicholas Anthony Vecchiarello, Charlottesville, VA (US); Arun Kannoth Nambiar, Cambrdge, MA (US); Glen Bolton, Boston, MA (US)

(73) Assignee: AMGEN INC., Thousand Oaks, CA (US)

(21) Appl. No.: 18/602,268

(22) Filed: Mar. 12, 2024

Related U.S. Application Data
(60) Provisional application No. 63/490,079, filed on Mar. 14, 2023.

(51) Int. Cl. C07K 1/18; B01D 15/36; B01D 15/38

(52) U.S. Cl. CPC C07K 1/18; B01D 15/363; B01D 15/3809

(57) ABSTRACT

Disclosed herein are methods for purifying a recombinant protein from a composition comprising the recombinant protein and at least one impurity, the methods comprising performing anion exchange chromatography (e.g., in flow-through or weak partitioning chromatography mode) using an anion exchange material comprising a primary amine ligand, such as a polyamine ligand.

### CROSS-REFERENCE TO RELATED APPLICATIONS

This application claims the benefit of priority to U.S. Provisional Patent Application No. 63/490,079, filed Mar. 14, 2023, which is hereby incorporated by reference in its entirety.

### FIELD

The present disclosure provides methods for purifying a recombinant protein from a composition comprising the recombinant protein and at least one impurity, the methods comprising performing anion exchange chromatography (e.g., in flow-through or weak partitioning chromatography mode) using an anion exchange material comprising a primary amine ligand, such as a polyamine ligand (e.g., TOYOPEARL® NH2-750F). In some embodiments, the composition has a pH of about 7.0 to about 8.0 and a conductivity of less than about 10 mS/cm (such as, e.g., about 3 mS/cm to about 7 mS/cm). Additionally, in some embodiments, the anion exchange chromatography unit operation enables robust high molecular weight species removal with high protein yield (such as, e.g., at least about 85%) at high loadings (such as, e.g., greater than about 100 g/L of anion exchange material).

### BACKGROUND

Downstream purification processes for drug substance manufacture of protein therapeutics such as monoclonal antibodies (mAbs) and antibody constructs generally include an affinity chromatography step followed by one or more polishing chromatography steps to remove product-related and process-related impurities. For products containing an Fc domain, a Protein A affinity chromatography step in which cell culture harvest fluid is flowed onto a Protein A resin to bind the recombinant protein of interest, followed by elution with a low pH buffer that desorbs the protein from the Protein A resin, is often used to capture the protein. Because the Protein A pool generally has low pH (≤pH 5), the subsequent unit operation is often a low pH viral inactivation (VI) step, in which the Protein A pool is titrated with an acid to a low pH known to inactivate enveloped viruses, held for a sufficient period to ensure VI, and then titrated with a base to a higher pH appropriate for product stability and/or loading onto subsequent unit operations. Many downstream purification processes utilize a cation exchange (CEX) chromatography step after the VI unit operation, as CEX generally requires a relatively low pH to bind positively-charged product and process-related impurities onto the negatively-charged CEX resin. Additional polishing chromatography steps, such as anion exchange (AEX) chromatography, hydrophobic interaction chromatography (HIC), and mixed mode chromatography (MMC), are often employed post-CEX to further reduce impurities. These polishing chromatography steps may be operated in various chromatographic modes depending on process requirements, including bind-and-elute mode, flow-through mode, and frontal loading (i.e., “frontal”) mode.

AEX chromatography can enable connected processing with downstream steps such as viral filtration (VF), which further reduces viral contamination risk, and ultrafiltration/diafiltration (UF/DF), which buffer exchanges and/or concentrates the product of interest to the desired conditions for drug product formulation. Moreover, flow-through and weak partitioning AEX chromatography generally allow for higher column loadings compared to chromatography operations run in bind-and-elute mode, which in turn reduces the required column sizes and buffer consumption in downstream processes. However, while flow-through AEX chromatography has demonstrated robustness for removal of process-related impurities such as nucleic acids, host cell proteins, leached protein A ligand, endotoxins, and viruses, achieving high molecular weight (HMW) species and aggregate removal can be challenging, especially when high protein loadings are used to maximize process productivity (Yigzaw et al, Current Pharmaceutical Biotechnology, 2009). Achieving robust HMW clearance requires optimization of the AEX resin, load pH, counter-ion type, and load dilution—variables subject to constraints such as product stability and facility fit.

Accordingly, there is a need in the art for new and improved purification methods utilizing AEX chromatography that enable robust HMW species removal with high protein yield at high loadings.

### SUMMARY

One aspect of the disclosure provides a method for purifying a recombinant protein from a composition comprising the recombinant protein and at least one impurity, the method comprising:
loading the composition onto an anion exchange material comprising a primary amine ligand at a loading density of greater than about 100 g/L of anion exchange material, wherein:
   the composition has a pH of about 7.0 to about 8.0 and a conductivity of less than about 10 mS/cm; and
   the at least one impurity binds to the anion exchange material more strongly than the recombinant protein binds to the anion exchange material; and collecting a purified composition comprising the recombinant protein.

Another aspect of the disclosure provides a method for purifying a recombinant protein (e.g., an antigen-binding protein, such as, e.g., an antibody) from a composition comprising the recombinant protein and at least one impurity (e.g., a high molecular weight species of the recombinant protein), the method comprising:
loading the composition onto an anion exchange material comprising resin particles, wherein the resin particles comprise polymethacrylate and are functionalized with a primary amine ligand, at a loading density of about 250 g/L to about 600 g/L of anion exchange material, wherein:
   the composition has a pH of about 7.0 to about 8.0 and a conductivity of less than about 10 mS/cm (e.g., about 3 mS/cm to about 6 mS/cm); and
   the at least one impurity binds to the anion exchange material more strongly than the recombinant protein binds to the anion exchange material; and
collecting a purified composition comprising the recombinant protein.

Still another aspect of the disclosure provides a method for purifying a recombinant protein (e.g., an antigen-binding protein, such as, e.g., an antibody) from a composition comprising the recombinant protein and at least one impurity (e.g., a high molecular weight species of the recombinant protein), the method comprising:
loading the composition onto an anion exchange material comprising a primary amine ligand at a loading density of about 250 g/L to about 600 g/L of anion exchange material, wherein:
   the composition has a pH of about 7.0 to about 8.0 and a conductivity of about 3 mS/cm to about 6 mS/cm; and
   the at least one impurity binds to the anion exchange material more strongly than the recombinant protein binds to the anion exchange material; and
collecting a purified composition comprising the recombinant protein, wherein:
   less than about 2.5% w/w of the recombinant protein in the purified composition is high molecular weight species of the recombinant protein; and/or
   the purified composition comprises at least about 85% w/w of the recombinant protein in the composition prior to the loading.

Yet another aspect of the disclosure provides a method for purifying a recombinant protein (e.g., an antigen-binding protein, such as, e.g., an antibody) from a composition comprising the recombinant protein and at least one impurity (e.g., a high molecular weight species of the recombinant protein), the method comprising:
loading the composition onto an anion exchange material comprising a polyamine ligand at a loading density of about 250 g/L to about 600 g/L of anion exchange material, wherein:
   the composition has a pH of about 7.0 to about 8.0 and a conductivity of about 3 mS/cm to about 6 mS/cm; and
   the at least one impurity binds to the anion exchange material more strongly than the recombinant protein binds to the anion exchange material; and collecting a purified composition comprising the recombinant protein, wherein:
   less than about 2.5% w/w of the recombinant protein in the purified composition is high molecular weight species of the recombinant protein; and/or
   the purified composition comprises at least about 85% w/w of the recombinant protein in the composition prior to the loading.

Another aspect of the disclosure provides a method for purifying a recombinant protein (e.g., an antigen-binding protein, such as, e.g., an antibody) from a composition comprising the recombinant protein and at least one impurity (e.g., a high molecular weight species of the recombinant protein), the method comprising:
performing a low pH viral inactivation unit operation employing formic acid as an acid titrant;
loading the composition onto an anion exchange material comprising a primary amine ligand at a loading density of about 250 g/L to about 600 g/L of anion exchange material, wherein:
   the composition has a pH of about 7.0 to about 8.0 and a conductivity of less than 10 mS/cm (such as, e.g., about 3 mS/cm to about 6 mS/cm);
   the at least one impurity binds to the anion exchange material more strongly than the recombinant protein binds to the anion exchange material; and
   the low pH viral inactivation unit operation is performed one or more unit operations prior to the loading; and
collecting a purified composition comprising the recombinant protein.

Still another aspect of the disclosure provides a method for purifying a recombinant protein (e.g., an antigen-binding protein, such as, e.g., an antibody) from a composition comprising the recombinant protein and at least one impurity selected from high molecular weight species of the recombinant protein, the method comprising:
performing a low pH viral inactivation unit operation employing about 1M to about 2M formic acid as an acid titrant;
loading the composition onto an anion exchange material comprising a polyamine ligand at a loading density of about 250 g/L to about 600 g/L of anion exchange material, wherein:
   the composition has a pH of about 7.0 to about 8.0 and a conductivity of less than 10 mS/cm (such as, e.g., about 3 mS/cm to about 6 mS/cm);
   the at least one impurity binds to the anion exchange material more strongly than the recombinant protein binds to the anion exchange material; and
   the low pH viral inactivation unit operation is performed one or more unit operations prior to the loading; and
collecting a purified composition comprising the recombinant protein.

Yet another aspect of the disclosure provides a method for purifying a recombinant protein (e.g., an antigen-binding protein, such as, e.g., an antibody) from a composition comprising the recombinant protein and at least one impurity (e.g., a high molecular weight species of the recombinant protein), the method comprising:
performing a low pH viral inactivation unit operation employing formic acid as an acid titrant;
loading the composition onto an anion exchange material comprising a primary amine ligand at a loading density of about 250 g/L to about 600 g/L of anion exchange material, wherein:
   the composition has a pH of about 7.0 to about 8.0 and a conductivity of about 3 mS/cm to about 6 mS/cm;
   the at least one impurity binds to the anion exchange material more strongly than the recombinant protein binds to the anion exchange material; and
   the low pH viral inactivation unit operation is performed one or more unit operations prior to the loading; and collecting a purified composition comprising the recombinant protein, wherein:
   less than about 2.5% w/w of the recombinant protein in the purified composition is high molecular weight species of the recombinant protein; and/or
   the purified composition comprises at least about 85% w/w of the recombinant protein in the composition prior to the loading.

### BRIEF DESCRIPTION OF THE DRAWINGS

FIG. 1A shows the percentage of high molecular weight (HMW) species of mAb1, as assessed by SEHPLC, in a composition loaded onto an AEX column and in a pool recovered from the AEX column across seven pilot-scale lots.

FIG. 1B shows step yield for the AEX step for the seven mAb1 pilot-scale lots summarized in FIG. 1A, demonstrating the ability of the AEX step to achieve high yields of greater than 85% while providing significant impurity reduction.

FIG. 2A shows the percentage of high molecular weight (HMW) species of mAb2, as assessed by SE-UHPLC, in a composition loaded onto an AEX column and in a pool recovered from the AEX column across two pilot-scale lots.

FIG. 2B shows step yield for the AEX step for the two mAb2 pilot-scale lots summarized in FIG. 2A, demonstrating the ability of the AEX step to achieve high yields of greater than 85% while providing significant impurity reduction.

FIG. 3 shows the percentage of high molecular weight (HMW) species of mAb1 in two pilot-scale lots, one in which 10% acetic acid was used as a VI titrant (PSL1) and one in which 1M formic acid was used as a VI titrant (PSL2). The use of a 1M formic acid VI titrant (PSL2) led to a lower HMW percentage in the AEX pool vs. the use of a 10% acetic acid VI titrant (PSL1) under similar conditions.

### DETAILED DESCRIPTION

Disclosed herein are methods for purifying a recombinant protein (e.g., an antigen-binding protein, such as, e.g., an antibody) from a composition comprising the recombinant protein and at least one impurity (e.g., a high molecular weight species of the recombinant protein), the methods comprising performing anion exchange chromatography (e.g., in flow-through or weak partitioning chromatography mode) using an anion exchange material comprising a primary amine ligand, such as a polyamine ligand, and optionally a methacrylate-containing polymer base matrix.

### Definitions

The following definitions are provided to assist in understanding the scope of this disclosure. Unless defined otherwise, all technical and scientific terms used herein have the same meaning as is commonly understood by one of ordinary skill in the art to which this disclosure belongs.

In some embodiments, “about,” when used in connection with a measurable numerical variable, refers to the indicated value of the variable and to all values of the variable that are within the experimental error of the indicated value (e.g., within the 95% confidence interval for the mean) or ±10% of the indicated value, whichever is greater. In some embodiments, numeric ranges are inclusive of the numbers defining the range (i.e., the endpoints).

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges also encompassed within the disclosure, subject to any specifically excluded limit in the stated range. Where the stated range includes one or both of the limits, ranges excluding either or both of those included limits are also included in the disclosure.

As used herein, the terms “a” and “an” mean “one or more” unless specifically indicated otherwise. Additionally, “one or more” and “at least one” are used interchangeably herein. Furthermore, unless otherwise required by context, singular terms include pluralities and plural terms include the singular.

As used herein, the term “acid precipitation” refers to a harvest operation in which cell culture pH is reduced to induce precipitation of one or more cell culture impurities.

As used herein, the term “affinity chromatography” (also referred to as “capture chromatography”) refers to a chromatography operation in which a biomolecule (e.g., a recombinant protein) is separated from a mixture based on a selective interaction between the biomolecule and another substance (i.e., a ligand). Affinity chromatography is commonly used in biomanufacturing processes to isolate and concentrate desired recombinant proteins from harvested cell culture fluid. In a typical affinity chromatography operation, a biomolecule in a moving phase selectively binds to or otherwise interacts with a stationary phase while the rest of the moving phase passes through the chromatography material. The biomolecule is then eluted from the stationary phase by changing the conditions in a manner that reduces the affinity between the ligand and the biomolecule. Non-limiting examples of affinity chromatography materials include Protein A, Protein G, Protein A/G, and Protein L materials. Additionally, immobilized metal affinity chromatography (IMAC) can be used to capture proteins that have or have been engineered to have affinity for metal ions.

In some embodiments, protein A affinity chromatography may be employed to capture a recombinant protein of interest. Protein A ligands are highly selective for a wide range of proteins containing an antibody Fc region and provide robust removal of process-related impurities with high target protein yields. Commercially available protein A materials include, but are not limited to, MABSELECT™ SURE Protein A, Protein A Sepharose FAST FLOW™ MABSELECT™ PrismA (Cytiva, Marborough, MA), PROSEP-A™ (Merck Millipore, U.K), TOYOPEARL® HC-650F Protein A (TosoHass Co., Philadelphia, PA), and AP Plus, Purolite, King of Prussia, PA).

As used herein, the term “antigen-binding protein” refers to a protein or polypeptide that comprises an antigen-binding region or antigen-binding portion that has affinity for another molecule to which it binds (antigen). Antigen-binding proteins include, but are not limited to, antibodies, fusion proteins, VH, VHH, VL, (s)dAb, Fv, light chain (VL-CL), Fd (VH-CH1), heavy chain, Fab, Fab′, F(ab′)2 or “r IgG” (“half antibody” consisting of a heavy chain and a light chain) or a modified antigen-binding portion of a full-length antibody, such as, e.g., a triple-chain antibody-like molecule, a heavy chain only antibody, single-chain variable fragment (scFv), di-scFv or bi(s) scFv, scFv-Fc, scFv-zipper, single-chain Fab (scFab), Fab₂, Fab₃, diabodies, single-chain diabodies, tandem diabodies (Tandabs), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies, such as triabodies or tetrabodies, and single domain antibodies, such as nanobodies or single variable domain antibodies comprising merely one variable region, which might be VHH, VH, or VL, that specifically binds to an antigen or target independently of other variable regions or domains.

As used herein, the term “antibody” generally refers to a tetrameric immunoglobulin protein comprising two light chain polypeptides (about 25 kDa each) and two heavy chain polypeptides (about 50-70 kDa each).

As used herein, the term “light chain” or “immunoglobulin light chain” refers to a polypeptide comprising, from amino terminus (N-terminus) to carboxyl terminus (C-terminus), a single immunoglobulin light chain variable region (VL) and a single immunoglobulin light chain constant domain (CL). The immunoglobulin light chain constant domain (CL) can be a human kappa (x) or human lambda (k) constant domain.

As used herein, the term “heavy chain” or “immunoglobulin heavy chain” refers to a polypeptide comprising, from amino terminus (N-terminus) to carboxyl terminus (C-terminus), a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e., between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein. From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001).

Papain digestion of antibodies produces two identical antigen-binding proteins, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

As used herein, a “Fc fragment” or “Fc region” of an immunoglobulin generally comprises two constant domains, a CH2 domain and a CH3 domain, and optionally comprises a CH4 domain. The Fc region may be an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as C1q binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function.

As used herein, the term “F(ab′)₂ fragment” refers to a bivalent fragment including two Fab′ fragments linked by a disulfide bridge between the heavy chains at the hinge region.

As used herein, the term “Fv” fragment refers to the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

As used herein, the term “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

As used herein, a “nanobody” refers to the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks, and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H₂L₂(referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure.

As used herein, the term “heavy chain-only antibody” refers to an immunoglobulin protein consisting of two heavy chain polypeptides (such as, e.g., heavy chain polypeptides that are about 50-70 kDa each). A “heavy chain-only antibody” lacks the two light chain polypeptides found in a conventional antibody. Heavy-chain antibodies constitute about one-fourth of the IgG antibodies produced by the camelids, e.g., camels and llamas (Hamers-Casterman C., et al. Nature. 363, 446-448 (1993)). These molecules are formed by two heavy chains but are devoid of light chains. As a consequence, the variable antigen binding part is referred to as the VHH domain, and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 3746 (1999)), and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)). Sharks have also been shown to have a single VH-like domain in their antibodies, termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003).)

In some embodiments, a “heavy chain-only antibody” is a dimeric antibody comprising a VH antigen-binding domain and the CH2 and CH3 constant domains, in the absence of the CH1 domain. In some embodiments, a heavy chain-only antibody is composed of a variable region antigen-binding domain composed of framework 1, CDR1, framework 2, CDR2, framework 3, CDR3, and framework 4. In some embodiments, a heavy chain-only antibody is composed of an antigen-binding domain, at least part of a hinge region, and CH2 and CH3 domains. In some embodiments, a heavy chain-only antibody is composed of an antigen-binding domain, at least part of a hinge region, and a CH2 domain. In some embodiments, a heavy chain-only antibody is composed of an antigen-binding domain, at least part of a hinge region, and a CH3 domain. Heavy chain-only antibodies in which the CH2 and/or CH3 domain is truncated are also included herein. The heavy chain-only antibodies described herein may belong to the IgG subclass, but heavy chain-only antibodies belonging to other subclasses, such as IgM, IgA, IgD and IgE subclass, are also included herein. In some embodiments, a heavy chain-only antibody may belong to the IgG1, IgG2, IgG3, or IgG4 subtype, e.g., the IgG1 or IgG4 subtype. In some embodiments, a heavy chain antibody-only is of the IgG1 or IgG4 subtype, wherein one or more of the CH domains is modified to alter an effector function of the antibody. In some embodiments, a heavy chain-only antibody is of the IgG4 subtype, wherein one or more of the CH domains is modified to alter an effector function of the antibody. In some embodiments, a heavy chain-only antibody is of the IgG1 subtype, wherein one or more of the CH domains is modified to alter an effector function of the antibody. Modifications of CH domains that alter effector function are further described herein. Non-limiting examples of heavy-chain-only antibodies are described, for example, in WO2018/039180, the disclosure of which is incorporated herein by reference herein in its entirety.

As used herein, the term “three-chain antibody like molecule” or “TCA” refers to an antibody-like molecule comprising, consisting essentially of, or consisting of three polypeptide subunits, two of which comprise, consist essentially of, or consist of one heavy and one light chain of a monoclonal antibody, or antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and one or more antigen binding domains (such as, e.g., two antigen binding domains) that binds an epitope of a second antigen or a different epitope of the first antigen, where such binding domain is derived from or has sequence identity with the variable region of an antibody heavy or light chain. Parts of such variable region may be encoded by VH and/or V_{L} gene segments, D and J_{H} gene segments, or J_{L}gene segments. The variable region may be encoded by rearranged V_{H}DJ_{H}, V_{L}DJ_{H}, V_{H}J_{L}, or V_{L}J_{L} gene segments.

As used herein, the term “bioreactor” means any vessel useful for the growth of a cell culture (e.g., a mammalian cell culture or a bacterial cell culture). “Bioreactor” encompasses the term “fermenter” (i.e., a vessel useful for the growth of a bacterial cell culture, which typically contains a more rigorous agitator and increased gas flow relative to a vessel used for the growth of a mammalian cell culture) herein. Non-limiting examples of bioreactors include stirred tank, airlift, fiber, microfiber, hollow fiber, ceramic matrix, fluidized bed, fixed bed, and/or spouted bed bioreactors. In some embodiments, an example bioreactor can perform one or more (e.g., one, two, three, all) of the following steps: feeding of nutrients and/or carbon sources, injection of suitable gas (such as, e.g., oxygen), inlet and outlet flow of fermentation or cell culture medium (e.g., perfusion of fresh cell culture medium in and removal of spent cell culture medium), separation of gas and liquid phases, maintenance of temperature, maintenance of oxygen and CO₂ levels, maintenance of pH level, agitation (e.g., stirring), and/or cleaning/sterilizing. Unless otherwise indicated by context, a bioreactor can be suitable for batch, semi fed-batch, fed-batch, perfusion, and/or continuous fermentation processes. Any suitable bioreactor diameter can be used. Unless otherwise indicated by context, in some embodiments, the bioreactor can have a volume between 100 mL and 50,000 L. Unless otherwise indicated, a bioreactor can be of any size so long as it is useful for the culturing of cells; typically, a bioreactor is sized appropriate to the volume of cell culture being grown inside of it. In non-limiting embodiments and unless otherwise indicated by context, a bioreactor may be at least 1 liter (L) or may be 2, 5, 10, 50, 100, 200, 250, 500, 1,000, 1500, 2000, 2,500, 5,000, 8,000, 10,000, 12,000 liters, 20,000 L or more, or any volume in between. The internal conditions of the bioreactor, including, but not limited to, pH, dissolved oxygen concentration, and temperature, can be controlled during the culturing period. Those of ordinary skill in the art will be aware of, and will be able to select, suitable bioreactors for use in the manufacturing methods disclosed herein based on the relevant considerations.

As used herein, the term “cell culture” or “culture” refers to the growth and propagation of cells outside of a multicellular organism or tissue. Suitable culture conditions for mammalian and bacterial cells are known in the art. (See, e.g., Animal cell culture: A Practical Approach, D. Rickwood, ed., Oxford University Press, New York (1992).) Mammalian cells may be cultured in suspension or while attached to a solid substrate. In some embodiments, fluidized bed bioreactors, hollow fiber bioreactors, roller bottles, shake flasks, and/or stirred tank bioreactors, with or without microcarriers, may be used for cell culture. In some embodiments, 500 L to 2000 L bioreactors are used for cell culture (e.g., as part of a seed train). In some embodiments, 1000 L to 2000 L bioreactors are used for cell culture (e.g., as part of a seed train).

As used herein, the term “cell culturing medium” (also referred to as “media,” “culture medium,” “cell culture media,” “tissue culture media,” and the like) refers to any nutrient solution used for growing cells, e.g., bacterial or mammalian cells. Cell culturing medium generally provides one or more of the following components: an energy source (e.g., in the form of a carbohydrate, such as, e.g., glucose); one or more essential amino acids (e.g., all essential amino acids; the twenty basic amino acids plus cysteine); vitamins and/or other organic compounds typically required at low concentrations; lipids or free fatty acids; and trace elements, such as, e.g., inorganic compounds or naturally occurring elements that are typically required at very low concentrations, such as, e.g., concentrations in the micromolar range. As used herein, cell culturing medium encompasses nutrient solutions that are typically employed in and/or are known for use with any cell culture process, including, but not limited to, batch, extended batch, fed-batch, intensified, and/or perfusion or continuous culturing of cells.

As used herein, the term “cell density” refers to the number of cells in a given volume of culture medium. “Viable cell density” refers to the number of live cells in a given volume of culture medium, as determined by standard viability assays (such as, e.g., a trypan blue dye exclusion method). As used herein, the term “packed cell volume” (PCV), also referred to as “percent packed cell volume” (% PCV), is the ratio of the volume occupied by the cells, to the total volume of cell culture, expressed as a percentage (see Stettler, et al., (2006) Biotechnol Bioeng. Dec. 20:95(6):1228-33). Packed cell volume is a function of cell density and cell diameter; increases in packed cell volume could arise from increases in cell density or cell diameter or both. Packed cell volume is a measure of the solid content in the cell culture. Since host cells vary in size and cell cultures also contain dead and dying cells and other cellular debris, packed cell volume can describe with a greater degree of accuracy the solid content within a cell culture.

As used herein, the term “connected,” in reference to unit operations, refers to a direct connection or mechanism that allows for continuous flow between one or more unit operations in a single operational cycle.

As used herein, the term “continuous,” in reference to unit operations, refers to a direct connection or mechanism that allows for continuous flow between one or more unit operations across multiple operational cycles.

As used herein, the term “dynamic binding capacity,” in reference to a chromatography material, refers to the amount of product, e.g., polypeptide, the material will bind under actual flow conditions before significant breakthrough of unbound product occurs.

As used herein, the term “expression vector” or “expression construct” refers to a recombinant DNA molecule containing a desired coding sequence and appropriate nucleic acid control sequences necessary for the expression of the operably linked coding sequence in a particular host cell, e.g., a mammalian host cell. Vectors can include viral vectors, nonepisomal mammalian vectors, plasmids, and other non-viral vectors. An expression vector can include sequences that affect or control transcription, translation, and, if introns are present, affect RNA splicing of a coding region operably linked thereto. “Operably linked” means that the components to which the term is applied are in a relationship that allows them to carry out their inherent functions. For example, a control sequence, e.g., a promoter, in a vector that is “operably linked” to a protein coding sequence are arranged such that normal activity of the control sequence leads to transcription of the protein coding sequence resulting in recombinant expression of the encoded protein.

As used herein, “fed-batch culture” refers to a form of suspension culture, specifically a method of culturing cells in which additional components are provided to the culture at a time or times subsequent to the beginning of the culture process. The provided components typically comprise nutritional supplements for the cells which have been depleted during the culturing process. Additionally or alternatively, the additional components may include supplementary components (such as, e.g., a cell-cycle inhibitory compound). In some embodiments, fed-batch cell culture medium formulations may be richer or more concentrated than basal cell culture medium formulations, which contain components essential for cell survival and growth and are typically used to initiate a cell culture. A fed-batch culture may be stopped at some point, and the cells and/or components in the medium may be harvested and optionally purified.

As used herein, a “fusion protein” is a protein that contains at least one polypeptide fused or linked to a heterologous polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell to produce the fusion protein. The fusion protein may comprise a fragment from an immunoglobulin protein, such as an Fc region, fused or linked to a ligand polypeptide, a receptor polypeptide, a hormone, cytokine, growth factor, an enzyme, or other polypeptide that is not a component of an immunoglobulin.

As used herein, a “growth phase” of a cell culture refers to the period of exponential cell growth (i.e., the log phase) where cells are generally rapidly dividing.

As used herein, the term “harvested cell culture fluid” refers to a solution which has been processed by one or more operations to separate cells, cell debris, or other large particulates from the recombinant protein. Such operations, as described herein, include, but are not limited to, cooling, flocculation, acidification, centrifugation, neutralization, acoustic wave separation, and various forms of filtration (e.g., depth filtration, microfiltration, ultrafiltration, tangential flow filtration, and alternating tangential flow filtration). Harvested cell culture fluid includes cell culture lysates as well as cell culture supernatants. The harvested cell culture fluid may be further clarified to remove fine particulate matter and soluble aggregates by filtration with a membrane having a pore size between about 0.1 μm and about 0.5 μm, such as, e.g., a membrane having a pore size of about 0.22 μm.

As used herein, a “host cell” refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises a nucleic acid encoding a recombinant protein, e.g., operably linked to at least one expression control sequence (e.g., promoter or enhancer), is a “recombinant host cell.” A host cell, when cultured under appropriate conditions, may synthesize a recombinant protein that can subsequently be collected from the culture medium (if the host cell secretes it into the medium) or directly from the host cell producing it (if it is not secreted).

As used herein, “high molecular weight” or “HMW” species of a recombinant protein of interest refer to dimers, oligomers, and aggregates of the recombinant protein that have a molecular weight greater than the molecular weight of the intact, fully assembled form of the recombinant protein.

As used herein, the term “impurity” refers to a component other than the recombinant protein of interest, along with its associated buffer components. Impurities include, but are not limited to, process- and product-related impurities, such as, e.g., host cell proteins, leached resin materials (such as, e.g., leached protein A), nucleic acids, HMW species of the recombinant protein, LMW species of the recombinant protein, endotoxins, viral contaminants, cell culture media components, and the like.

As used herein, the term “loading density” refers to the amount of composition put in contact with a volume of chromatography material.

As used herein, “low molecular weight” or “LMW” species of a recombinant protein of interest refer to fragments, truncated forms, or other incomplete variants of the recombinant protein that have a molecular weight less than the molecular weight of the intact, fully assembled form of the recombinant protein. LMW species can include, but are not limited to, proteolytic fragments, truncated forms resulting from cellular expression of mRNA splice variants, and single component polypeptides in the case of multi-polypeptide chain proteins (such as, e.g., light chain or heavy chain only species when the recombinant protein is an antibody).

As used herein, a “perfusion” cell culture medium refers to a cell culture medium that is typically used in cell cultures that are maintained by perfusion or continuous culture methods and is sufficiently complete to support the cell culture during this process. In some embodiments, perfusion cell culture medium formulations may be richer or more concentrated than base cell culture medium formulations to accommodate the method used to remove the spent medium. In some embodiments, perfusion cell culture medium may be used during both the growth and production phases.

As used herein, the term “polishing chromatography” refers to a chromatography operation performed after a capture or affinity chromatography operation to remove remaining impurities and obtain a more highly purified composition and/or recombinant protein. Common impurities removed during polishing steps include, but are not limited to, product-related impurities (e.g., HMW and LMW species), host cell proteins, DNA, leached protein A, viral contaminants, and endotoxins. In addition, typical chromatography techniques used for polishing include, but are not limited to, ion exchange chromatography (IEX), hydrophobic interaction chromatography (HIC), and multimodal (or mixed mode) chromatography (MMC).

As used herein, “anion exchange chromatography” (AEX) refers to a form of ion exchange chromatography performed on a solid phase medium (e.g., resin or membrane) that is positively charged and has the capacity to exchange free anions with anions in an aqueous solution passed over or through the solid phase. AEX chromatography is used, for example, for viral clearance and impurity removal. Commercially available anion exchange media include, but are not limited to, sulphopropyl (SP) immobilized on agarose (e.g., Source 15 Q, Capto™ Q Q-SEPHAROSE FAST FLOW™ (Cytiva), FRACTOGEL EMD TMAE™, FRACTOGEL EMD DEAE™, (EMD Merck), TOYOPEARL® Super Q® and TOYOPEARL® NH2-750F (Tosoh Bioscience), POROS HQ™, and POROS XQ™, (ThermoFisher).

As used herein, “cation exchange chromatography” (CEX) refers to a form of ion exchange chromatography performed on a solid phase medium (e.g., resin or membrane) that is negatively charged and has the capacity to exchange free cations with cations in an aqueous solution passed over or through the solid phase. The charge may be provided by attaching one or more charged ligands to the solid phase, e.g., via covalent linkage. Alternatively or additionally, the charge may be an inherent property of the solid phase (e.g., silica, which has an overall negative charge). CEX chromatography is typically used to remove high molecular weight (HMW) contaminants, process related impurities, and/or viral contaminants. Commercially available cation exchange media include, but are not limited to, sulphopropyl (SP) immobilized on agarose (e.g., SPSEPHAROSE FAST FLOW™, SP-SEPHAROSE FAST FLOW XL™ or SP-SEPHAROSE HIGH PERFORMANCE™, CAPTO S™, CAPTO SP ImpRes™, CAPTO S ImpAct™ (Cytiva), FRACTOGEL-SO3™, FRACTOGEL-SE HICAP™, and FRACTOPREP™ (EMD Merck, Darmstadt, Germany), TOYOPEARL® XS, TOYOPEARL® HS (Tosoh Bioscience, King of Prussia, PA), UNOsphere™ (BioRad, Hercules, CA), S Ceramic Hyper™ DF (Pall, Port Washington, NY), POROS™ (ThermoFisher, Waltham, MA), ESHMUNO® CSP and ESHMUNO® CP-FT (Millipore Sigma, Darmstadt, Germany).

As used herein, “hydrophobic interaction chromatography” (HIC) refers to chromatography performed on a solid phase medium that makes use of the interaction between hydrophobic ligands and hydrophobic residues on the surface of a desired solute (e.g., a desired protein). Commercially available hydrophobic interaction chromatography media include, but are not limited to, Phenyl Sephrose™ (Cytiva), Tosoh hexyl (Tosoh Bioscience), and Capto™ phenyl (Cytiva).

As used herein, “mixed-mode or multi-modal chromatography” (MMC) refers to chromatography that makes use of more than one form of interaction between the stationary phase and analyte to achieve separation. MMC differs from single mode chromatography in that two or more interaction types, such as, e.g., electrostatic, hydrogen bonding, and/or hydrophobic interactions, contribute significantly to the retention of solutes. Commercially available multi-modal chromatography media include, but are not limited to, Capto™ Adhere, Capto™ MMC Impress, Capto MMC, (Cytiva), PPA Hypercel, MEP Hypercell, HEA Hypercell (Pall Corporation, Port Washington, NY). Eshmuno HCX, (Merk Millipore), and TOYOPEARL® MX-Trp-650M (Tosoh Bioscience).

Polishing chromatography unit operations make use of materials (e.g., resins and/or membranes) containing agents that can be operated in a variety of modes, including bind-and-elute mode and flow-though mode. In bind-and-elute chromatography, a biomolecule of interest is usually loaded onto the chromatography material to maximize dynamic binding capacity and then specified recovery and elution conditions are utilized to maximize product purity in the eluate. By contrast, in flow-through chromatography, load conditions are employed that allow impurities to bind to the chromatography material while the biomolecule of interest passes through. Relative to bind-and-elute chromatography, flow-through chromatography allows for higher load densities for many biomolecules.

In addition to the two most common modes, weak partitioning chromatography, overload chromatography, and frontal chromatography modes may also be employed in purification processes. In weak partitioning chromatography, an isocratic separation method, flow-through mode is altered by identifying solution conditions that promote weak binding of a biomolecule to resin (Kₚ is about 0.1 to about 100, compared to a Kₚ of less than about 0.1 for flow-through chromatography), in addition to binding of one or more impurities. In overload chromatography, the biomolecule of interest is loaded onto the chromatography material beyond the dynamic binding capacity of the material. Additionally, frontal chromatography mode allows for a continuous, high-density feed (containing the protein of interest and at least one impurity) onto the chromatography medium. In frontal chromatography, the separation of the protein of interest from impurities and contaminants is driven by the binding affinity of the components in the load feed for the chromatography medium. The amount of the protein of interest that may be loaded on and bound to the chromatography medium in frontal mode is typically dependent on the amount of more highly charged impurities/contaminants, such as product-related impurities, in the load feed. Initially, all the components in the load feed will bind to the chromatography medium. Separation of the product of interest from the impurities/contaminants is driven by affinity for the chromatography medium. When the chromatography medium reaches saturation binding, those components in the load feed having a greater affinity for the chromatography medium (typically product-related impurities such as a HMW species) will displace proteins having a weaker affinity (e.g., the product of interest) resulting in the separation of the proteins with weaker affinity from the chromatography medium. These proteins exit the column in the load flow through as a band. As loading proceeds, the bound proteins are continuously displaced in order of increasing affinity for the chromatography medium until the column is at or near saturation with proteins having greater affinity than the protein of interest.

As used herein, the term “polypeptide” refers to a polymer of amino acids comprising at least 50 amino acids, such as, e.g., at least 100 amino acids.

As used herein, the term “partition coefficient” or “product partition coefficient” (Kₚ) refers to the molar concentration of product, e.g., recombinant protein, bound to the stationary phase divided by the molar concentration of the product in the mobile phase during a chromatography step.

As used herein, a “production” cell culture medium refers to a cell culture medium that is typically used in a cell culture during the transition when exponential growth is ending and protein production takes over (i.e., “transition” and/or “product” phases) and is sufficiently complete to maintain a desired cell density, viability, and/or product titer during this phase. A production cell culture medium may be the same as or different than the cell culture medium used during the exponential growth phase of the cell culture.

As used herein, a “production phase” of a cell culture refers to the period of time during which logarithmic cell growth has ended and recombinant protein production is predominant.

As used herein, the term “recombinant protein” refers to a heterologous protein produced by a host cell transfected with a nucleic acid encoding the protein when the host cell is cultivated in cell culture.

As used herein, the term “purified,” when used in relation to a composition, refers to a composition wherein at least one impurity is present at a lower concentration in the purified composition relative to the composition as it existed prior to one or more unit operations. Additionally, a “purified” recombinant protein (e.g., a purified antibody) refers to a recombinant purity which has been increased in purity, such that it exists in a form that is more pure than it exists in its natural environment and/or when initially synthesized and/or amplified under laboratory conditions. Purity is a relative term and does not necessarily refer to absolute purity.

As used herein, the term “titrant” refers to a solution of known concentration that is added to another solution during a titration. An “acid titrant” refers to a titrant with a pH of less than about 7.

As used herein, the term “unit operation” refers to a functional step that is performed as part of a process of purifying a recombinant protein of interest. Unit operations can be designed to achieve a single objective or multiple objectives, such as capture, acid precipitation, centrifugation, or chromatography steps. Unit operations can also include holding or storing steps between processing steps.

### Non-Limiting Example Features

Without limitation, some example embodiments/features of the present disclosure include E1-E47:

E1. A method for purifying a recombinant protein from a composition comprising the recombinant protein and at least one impurity, comprising:
loading the composition onto an anion exchange material comprising a primary amine ligand at a loading density of greater than about 100 g/L of anion exchange material, wherein:
   the composition has a pH of about 7.0 to about 8.0 and a conductivity of less than about 10 mS/cm; and
   the at least one impurity binds to the anion exchange material more strongly than the recombinant protein binds to the anion exchange material; and
collecting a purified composition comprising the recombinant protein.
   E2. The method according to E1, wherein the anion exchange material comprises resin particles.
   E3. The method according to E1 or E2, wherein the anion exchange material comprises resin particles, wherein at least about 80% of the resin particles have a particle size of about 30 μm to about 60 μm.
   E4. The method according to any one of E1-E3, wherein the anion exchange material comprises resin particles with an average particle size of about 45 μm.
   E5. The method according to any one of E1-E4, wherein the anion exchange material comprises a polyamine ligand.
   E6. The method according to any one of E1-E5, wherein the anion exchange material comprises a methacrylate-containing polymer base matrix.
   E7. The method according to any one of E1-E6, wherein the anion exchange material is TOYOPEARL® NH2-750F.
   E8. The method according to any one of E1-E7, wherein the loading density is less than about 600 g/L of anion exchange material.
   E9. The method according to any one of E1-E7, wherein the loading density is about 200 g/L to about 600 g/L of anion exchange material.
   E10. The method according to any one of E1-E9, wherein the composition has a conductivity of about 3 mS/cm to about 6 mS/cm.
   E11. The method according to any one of E1-E10, wherein the partition coefficient of the anion exchange material for the recombinant protein is about 0.1 to about 100.
   E12. The method according to any one of E1-E10, wherein the partition coefficient of the anion exchange material for the recombinant protein is about 20 to about 40.
   E13. The method according to any one of E1-E12, wherein the method comprises using an equilibration buffer and/or a recovery buffer with the anion exchange material, wherein:
the pH of the equilibration buffer and/or the recovery buffer is about 7.0 to about 8.0; and/or
the conductivity of the equilibration buffer and/or the recovery buffer is less than about 10 mS/cm.
   E14. The method according to E13, wherein the conductivity of the equilibration buffer and/or the recovery buffer is about 2 mS/cm to about 4 mS/cm.
   E15. The method according to any one of E1-E14, further comprising performing a low pH viral inactivation unit operation prior to the loading (e.g., one or more unit operations prior to the loading).
   E16. The method according to E15, wherein the low pH viral inactivation unit operation is performed at a pH of about 3.5 to about 3.7.
   E17. The method according to E15 or E16, wherein the low pH viral inactivation unit operation employs an acid titrant.
   E18. The method according to E17, wherein the acid titrant comprises formic acid.
   E19. The method according to E17 or E18, wherein the acid titrant is about 1M to about 2M formic acid (e.g., about 1M formic acid; about 2M formic acid).
   E20. The method according to any one of E15-E19, wherein the low pH viral inactivation unit operation is performed for at least about 60 minutes.
   E21. The method according to any one of E15-E20, wherein the low pH viral inactivation unit operation is performed for about 60 minutes to about 12 hours.
   E22. The method according to any one of E1-E21, further comprising performing one or more additional chromatography unit operations.
   E23. The method according to E22, wherein the one or more additional chromatography unit operations comprises an affinity chromatography unit operation performed prior to the loading.
   E24. The method according to E23, wherein the affinity chromatography unit operation is selected from protein A chromatography, protein G chromatography, protein L chromatography, and CH1 domain chromatography.
   E25. The method according to E23 or E24, wherein the affinity chromatography unit operation is protein A chromatography.
   E26. The method according to any one of E22-E25, wherein the one or more additional chromatography unit operations comprises an additional polishing chromatography unit operation performed prior to the loading.
   E27. The method according to any one of E22-E25, wherein the one or more additional chromatography unit operations comprises an additional polishing chromatography unit operation performed after the loading.
   E28. The method according to E26 or E27, wherein the additional polishing chromatography unit operation is selected from cation exchange chromatography, hydrophobic interaction chromatography, and mixed mode chromatography.
   E29. The method according to any one of E26-E28, wherein the additional polishing chromatography unit operation and the loading are continuous or connected.
   E30. The method according to any one of E1-E29, further comprising performing a viral filtration unit operation and/or a UF/DF unit operation after the loading.
   E31. The method according to E30, wherein the loading and the viral filtration unit operation and/or the UF/DF unit operation are continuous or connected.
   E32. The method according to any one of E1-E31, wherein less than about 5% w/w of the recombinant protein in the purified composition is high molecular weight species of the recombinant protein.
   E33. The method according to any one of E1-E32, wherein less than about 2.5% w/w of the recombinant protein in the purified composition is high molecular weight species of the recombinant protein.
   E34. The method according to any one of E1-E33, wherein less than about 1% w/w of the recombinant protein in the purified composition is high molecular weight species of the recombinant protein.
   E35. The method according to any one of E1-E34, wherein the purified composition comprises at least about 85% w/w of the recombinant protein in the composition prior to the loading.
   E36. The method according to any one of E1-E35, wherein the purified composition comprises at least about 90% w/w of the recombinant protein in the composition prior to the loading.
   E37. The method according to any one of E1-E36, wherein:
less than about 1% w/w of the recombinant protein in the purified composition is high molecular weight species of the recombinant protein; and
the purified composition comprises at least about 90% w/w of the recombinant protein in the composition prior to the loading.
   E38. The method according to any one of E1-E37, wherein:
the loading density is about 100 g/L to about 600 g/L of anion exchange material;
less than about 1% w/w of the recombinant protein in the purified composition is high molecular weight species of the recombinant protein; and
the purified composition comprises at least about 90% w/w of the recombinant protein in the composition prior to the loading.
   E39. The method according to any one of E1-E38, wherein the recombinant protein is an antigen-binding protein.
   E40. The method according to any one of E1-E39, wherein the recombinant protein is an antibody.
   E41. The method according to any one of E1-E40, wherein the at least one impurity is selected from host cell proteins, nucleic acids, high molecular weight species of the recombinant protein, fragments of the recombinant protein, cell culture media components, and viral contaminants.
   E42. The method according to any one of E1-E41, wherein the at least one impurity is selected from high molecular weight species of the recombinant protein.
   E43. The method according to any one of E1-E42, wherein the anion exchange material comprises resin particles, wherein the resin particles comprise polymethacrylate and are functionalized with a primary amine ligand.
   E44. The method according to any one of E1-E42, wherein the anion exchange material comprises resin particles, wherein the resin particles comprise a hydroxylated methacrylic polymer and are functionalized with a primary amine ligand.
   E45. The method according to E43 or E44, wherein at least about 80% of the resin particles have a particle size of about 30 μm to about 60 μm.
   E46. The method according to any one of E43, E44, or E45, wherein the resin particles have an average particle size of about 40 μm to about 50 μm.
   E47. The method according to E46, wherein the resin particles have an average particle size of about 45 μm.

### Anion Exchange Chromatography Purification Methods

Provided herein is a method for purifying a recombinant protein from a composition comprising the recombinant protein and at least one impurity, the method comprising:
loading the composition onto an anion exchange material comprising a primary amine ligand at a loading density of greater than about 100 g/L of anion exchange material, wherein:
   the composition has a pH of about 7.0 to about 8.0 and a conductivity of less than about 10 mS/cm; and
   the at least one impurity binds to the anion exchange material more strongly than
the recombinant protein binds to the anion exchange material; and
collecting a purified composition comprising the recombinant protein.

In some embodiments, the anion exchange material comprises resin particles.

In some embodiments, the anion exchange material comprises resin particles, wherein at least about 80% of the resin particles have a particle size of about 30 μm to about 60 μm.

In some embodiments, the anion exchange material comprises resin particles with an average particle size of about 30 μm to about 60 μm. In some embodiments, the anion exchange material comprises resin particles with an average particle size of about 40 μm to about 50 μm. In some embodiments, the anion exchange material comprises resin particles with an average particle size of about 30 μm. In some embodiments, the anion exchange material comprises resin particles with an average particle size of about 35 μm. In some embodiments, the anion exchange material comprises resin particles with an average particle size of about 40 μm. In some embodiments, the anion exchange material comprises resin particles with an average particle size of about 45 μm. In some embodiments, the anion exchange material comprises resin particles with an average particle size of about 50 μm. In some embodiments, the anion exchange material comprises resin particles with an average particle size of about 55 μm. In some embodiments, the anion exchange material comprises resin particles with an average particle size of about 60 μm.

In some embodiments, the anion exchange material comprises a polyamine ligand.

In some embodiments, the anion exchange material comprises a methacrylate-containing polymer base matrix.

In some embodiments, the anion exchange material comprises a methacrylate-containing polymer base matrix and a polyamine ligand.

In some embodiments, the anion exchange material comprises resin particles, wherein the resin particles comprise polymethacrylate and are functionalized with a primary amine ligand. In some embodiments, the anion exchange material comprises resin particles, wherein the resin particles comprise polymethacrylate and are functionalized with a primary amine ligand, and further wherein at least about 80% of the resin particles have a particle size of about 30 μm to about 60 μm. In some embodiments, the anion exchange material comprises resin particles, wherein the resin particles comprise polymethacrylate and are functionalized with a primary amine ligand, and further wherein the resin particles have an average particle size of about 40 μm to about 50 μm (such as, e.g., about 45 μm).

In some embodiments, the anion exchange material comprises resin particles, wherein the resin particles comprise a hydroxylated methacrylic polymer and are functionalized with a primary amine ligand. In some embodiments, the anion exchange material comprises resin particles, wherein the resin particles comprise a hydroxylated methacrylic polymer and are functionalized with a primary amine ligand, and further wherein at least about 80% of the resin particles have a particle size of about 30 μm to about 60 μm. In some embodiments, the anion exchange material comprises resin particles, wherein the resin particles comprise a hydroxylated methacrylic polymer and are functionalized with a primary amine ligand, and further wherein the resin particles have an average particle size of about 40 μm to about 50 μm (such as, e.g., about 45 μm).

In some embodiments, the anion exchange material is TOYOPEARL® NH2-750F.

In some embodiments, the loading density is less than about 750 g/L of anion exchange material. In some embodiments, the loading density is less than about 700 g/L of anion exchange material. In some embodiments, the loading density is less than about 650 g/L of anion exchange material. In some embodiments, the loading density is less than about 600 g/L of anion exchange material. In some embodiments, the loading density is less than about 550 g/L of anion exchange material. In some embodiments, the loading density is less than about 500 g/L of anion exchange material. In some embodiments, the loading density is less than about 450 g/L of anion exchange material. In some embodiments, the loading density is less than about 400 g/L of anion exchange material. In some embodiments, the loading density is less than about 350 g/L of anion exchange material. In some embodiments, the loading density is less than about 300 g/L of anion exchange material. In some embodiments, the loading density is less than about 250 g/L of anion exchange material. In some embodiments, the loading density is less than about 200 g/L of anion exchange material. In some embodiments, the loading density is less than about 150 g/L of anion exchange material.

In some embodiments, the loading density is about 100 g/L to about 600 g/L of anion exchange material. In some embodiments, the loading density is about 150 g/L to about 600 g/L of anion exchange material. In some embodiments, the loading density is about 200 g/L to about 600 g/L of anion exchange material. In some embodiments, the loading density is about 250 g/L to about 600 g/L of anion exchange material.

In some embodiments, the composition has a pH of about 7.1 to about 7.9. In some embodiments, the composition has a pH of about 7.2 to about 7.8. In some embodiments, the composition has a pH of about 7.3 to about 7.7. In some embodiments, the composition has a pH of about 7.4 to about 7.6. In some embodiments, the composition has a pH of about 7.5.

In some embodiments, the composition has a conductivity of less than about 9 mS/cm. In some embodiments, the composition has a conductivity of less than about 8 mS/cm. In some embodiments, the composition has a conductivity of less than about 7 mS/cm. In some embodiments, the composition has a conductivity of less than about 6 mS/cm. In some embodiments, the composition has a conductivity of less than about 5 mS/cm. In some embodiments, the composition has a conductivity of less than about 4 mS/cm.

In some embodiments, the composition has a conductivity of about 10 mS/cm. In some embodiments, the composition has a conductivity of about 9.5 mS/cm. In some embodiments, the composition has a conductivity of about 9 mS/cm. In some embodiments, the composition has a conductivity of about 8.5 mS/cm. In some embodiments, the composition has a conductivity of about 8 mS/cm. In some embodiments, the composition has a conductivity of about 7.5 mS/cm. In some embodiments, the composition has a conductivity of about 7 mS/cm. In some embodiments, the composition has a conductivity of about 6.5 mS/cm. In some embodiments, the composition has a conductivity of about 6 mS/cm. In some embodiments, the composition has a conductivity of about 5.5 mS/cm. In some embodiments, the composition has a conductivity of about 5 mS/cm. In some embodiments, the composition has a conductivity of about 4.5 mS/cm. In some embodiments, the composition has a conductivity of about 4 mS/cm. In some embodiments, the composition has a conductivity of about 3.5 mS/cm. In some embodiments, the composition has a conductivity of about 3 mS/cm.

In some embodiments, the composition has a conductivity of about 3 mS/cm to about 7 mS/cm. In some embodiments, the composition has a conductivity of about 3 mS/cm to about 6 mS/cm. In some embodiments, the composition has a conductivity of about 3 mS/cm to about 5 mS/cm.

In some embodiments, the partition coefficient of the anion exchange material for the recombinant protein is less than about 0.1. In some embodiments, the partition coefficient of the anion exchange material for the recombinant protein is less than about 10. In some embodiments, the partition coefficient of the anion exchange material for the recombinant protein is less than about 20. In some embodiments, the partition coefficient of the anion exchange material for the recombinant protein is less than about 30. In some embodiments, the partition coefficient of the anion exchange material for the recombinant protein is less than about 40. In some embodiments, the partition coefficient of the anion exchange material for the recombinant protein is less than about 50. In some embodiments, the partition coefficient of the anion exchange material for the recombinant protein is less than about 60. In some embodiments, the partition coefficient of the anion exchange material for the recombinant protein is less than about 70. In some embodiments, the partition coefficient of the anion exchange material for the recombinant protein is less than about 80. In some embodiments, the partition coefficient of the anion exchange material for the recombinant protein is less than about 90. In some embodiments, the partition coefficient of the anion exchange material for the recombinant protein is less than about 100.

In some embodiments, the partition coefficient of the anion exchange material for the recombinant protein is about 0.1 to about 100. In some embodiments, the partition coefficient of the anion exchange material for the recombinant protein is about 10 to about 100. In some embodiments, the partition coefficient of the anion exchange material for the recombinant protein is about 20 to about 100. In some embodiments, the partition coefficient of the anion exchange material for the recombinant protein is about 20 to about 90. In some embodiments, the partition coefficient of the anion exchange material for the recombinant protein is about 20 to about 80. In some embodiments, the partition coefficient of the anion exchange material for the recombinant protein is about 20 to about 70. In some embodiments, the partition coefficient of the anion exchange material for the recombinant protein is about 20 to about 60. In some embodiments, the partition coefficient of the anion exchange material for the recombinant protein is about 20 to about 50. In some embodiments, the partition coefficient of the anion exchange material for the recombinant protein is about 20 to about 40.

In some embodiments, the method comprises using an equilibration buffer and/or a recovery buffer with the anion exchange material, wherein:
the pH of the equilibration buffer and/or the recovery buffer is about 7.0 to about 8.0; and/or
the conductivity of the equilibration buffer and/or the recovery buffer is less than about 10 mS/cm.

In some embodiments, the equilibration buffer and/or the recovery buffer has a pH of about 7.1 to about 7.9. In some embodiments, the equilibration buffer and/or the recovery buffer has a pH of about 7.2 to about 7.8. In some embodiments, the equilibration buffer and/or the recovery buffer has a pH of about 7.3 to about 7.7. In some embodiments, the equilibration buffer and/or the recovery buffer has a pH of about 7.4 to about 7.6. In some embodiments, the equilibration buffer and/or the recovery buffer has a pH of about 7.5.

In some embodiments, the equilibration buffer and/or the recovery buffer has a conductivity of less than about 9 mS/cm. In some embodiments, the equilibration buffer and/or the recovery buffer has a conductivity of less than about 8 mS/cm. In some embodiments, the equilibration buffer and/or the recovery buffer has a conductivity of less than about 7 mS/cm. In some embodiments, the equilibration buffer and/or the recovery buffer has a conductivity of less than about 6 mS/cm. In some embodiments, the equilibration buffer and/or the recovery buffer has a conductivity of less than about 5 mS/cm. In some embodiments, the equilibration buffer and/or the recovery buffer has a conductivity of less than about 4 mS/cm. In some embodiments, the equilibration buffer and/or the recovery buffer has a conductivity of less than about 3 mS/cm. In some embodiments, the equilibration buffer and/or the recovery buffer has a conductivity of less than about 2 mS/cm.

In some embodiments, the equilibration buffer and/or the recovery buffer has a conductivity of about 10 mS/cm. In some embodiments, the equilibration buffer and/or the recovery buffer has a conductivity of about 9.5 mS/cm. In some embodiments, the equilibration buffer and/or the recovery buffer has a conductivity of about 9 mS/cm. In some embodiments, the equilibration buffer and/or the recovery buffer has a conductivity of about 8.5 mS/cm. In some embodiments, the equilibration buffer and/or the recovery buffer has a conductivity of about 8 mS/cm. In some embodiments, the equilibration buffer and/or the recovery buffer has a conductivity of about 7.5 mS/cm. In some embodiments, the equilibration buffer and/or the recovery buffer has a conductivity of about 7 mS/cm. In some embodiments, the equilibration buffer and/or the recovery buffer has a conductivity of about 6.5 mS/cm. In some embodiments, the equilibration buffer and/or the recovery buffer has a conductivity of about 6 mS/cm. In some embodiments, the equilibration buffer and/or the recovery buffer has a conductivity of about 5.5 mS/cm. In some embodiments, the equilibration buffer and/or the recovery buffer has a conductivity of about 5 mS/cm. In some embodiments, the equilibration buffer and/or the recovery buffer has a conductivity of about 4.5 mS/cm. In some embodiments, the equilibration buffer and/or the recovery buffer has a conductivity of about 4 mS/cm. In some embodiments, the equilibration buffer and/or the recovery buffer has a conductivity of about 3.5 mS/cm. In some embodiments, the equilibration buffer and/or the recovery buffer has a conductivity of about 3 mS/cm. In some embodiments, the equilibration buffer and/or the recovery buffer has a conductivity of about 2.5 mS/cm. In some embodiments, the equilibration buffer and/or the recovery buffer has a conductivity of about 2 mS/cm. In some embodiments, the equilibration buffer and/or the recovery buffer has a conductivity of about 1.5 mS/cm. In some embodiments, the equilibration buffer and/or the recovery buffer has a conductivity of about 1 mS/cm.

In some embodiments, the equilibration buffer and/or the recovery buffer has a conductivity of about 1 mS/cm to about 6 mS/cm. In some embodiments, the equilibration buffer and/or the recovery buffer has a conductivity of about 1 mS/cm to about 5 mS/cm. In some embodiments, the equilibration buffer and/or the recovery buffer has a conductivity of about 1 mS/cm to about 4 mS/cm. In some embodiments, the equilibration buffer and/or the recovery buffer has a conductivity of about 1 mS/cm to about 3 mS/cm.

In some embodiments, the equilibration buffer and/or the recovery buffer has a conductivity of about 2 mS/cm to about 6 mS/cm. In some embodiments, the equilibration buffer and/or the recovery buffer has a conductivity of about 2 mS/cm to about 5 mS/cm. In some embodiments, the equilibration buffer and/or the recovery buffer has a conductivity of about 2 mS/cm to about 4 mS/cm. In some embodiments, the equilibration buffer and/or the recovery buffer has a conductivity of about 2 mS/cm to about 3 mS/cm.

In some embodiments, the method further comprises performing a low pH viral inactivation unit operation prior to the loading (e.g., one or more unit operations prior to the loading). In some embodiments, the low pH viral inactivation unit operation is performed at a pH of about 3.5 to about 3.7. In some embodiments, the low pH viral inactivation unit operation is performed at a pH of about 3.5. In some embodiments, the low pH viral inactivation unit operation is performed at a pH of about 3.6. In some embodiments, the low pH viral inactivation unit operation is performed at a pH of about 3.7.

In some embodiments, the low pH viral inactivation unit operation employs an acid titrant. In some embodiments, the acid titrant is formic acid. In some embodiments, the acid titrant is about 1M to about 2M formic acid. In some embodiments, the acid titrant is about 1M formic acid. In some embodiments, the acid titrant is about 2M formic acid.

In some embodiments, the low pH viral inactivation unit operation is performed for at least about 60 minutes. In some embodiments, the low pH viral inactivation unit operation is performed for at least about 2 hours. In some embodiments, the low pH viral inactivation unit operation is performed for at least about 3 hours. In some embodiments, the low pH viral inactivation unit operation is performed for at least about 4 hours. In some embodiments, the low pH viral inactivation unit operation is performed for at least about 5 hours. In some embodiments, the low pH viral inactivation unit operation is performed for at least about 6 hours.

In some embodiments, the low pH viral inactivation unit operation is performed for at least about 7 hours. In some embodiments, the low pH viral inactivation unit operation is performed for at least about 8 hours. In some embodiments, the low pH viral inactivation unit operation is performed for about 60 minutes to about 12 hours. In some embodiments, the low pH viral inactivation unit operation is performed for about 60 minutes to about 8 hours.

In some embodiments, the method further comprises performing one or more additional chromatography unit operations.

In some embodiments, the one or more additional chromatography unit operations comprises an affinity chromatography unit operation performed prior to the loading. In some embodiments, the affinity chromatography unit operation is selected from protein A chromatography, protein G chromatography, protein L chromatography, and CH1 domain chromatography. In some embodiments, the affinity chromatography unit operation is protein A chromatography. In some embodiments, the affinity chromatography unit operation is protein G chromatography. In some embodiments, the affinity chromatography unit operation is protein L chromatography. In some embodiments, the affinity chromatography unit operation is CH1 domain chromatography.

In some embodiments, the one or more additional chromatography unit operations comprises an additional polishing chromatography unit operation performed prior to the loading. In some embodiments, the one or more additional chromatography unit operations comprises an additional polishing chromatography unit operation performed after the loading. In some embodiments, the additional polishing chromatography unit operation and the loading are connected. In some embodiments, the additional polishing chromatography unit operation and the loading are not connected. In some embodiments, the additional polishing chromatography unit operation and the loading are continuous. In some embodiments, the additional polishing chromatography unit operation and the loading are not continuous.

In some embodiments, the additional polishing chromatography unit operation is selected from cation exchange chromatography, hydrophobic interaction chromatography, and mixed mode chromatography. In some embodiments, the additional polishing chromatography unit operation is cation exchange chromatography. In some embodiments, the additional polishing chromatography unit operation is hydrophobic interaction chromatography. In some embodiments, the additional polishing chromatography unit operation is mixed mode chromatography.

In some embodiments, the method further comprises performing a viral filtration unit operation and/or a UF/DF unit operation after the loading.

In some embodiments, less than about 5% w/w (e.g., less than about 4.5% w/w, less than about 4% w/w, less than about 3.5% w/w, less than about 3% w/w, less than about 2.5% w/w, less than 2% w/w, less than about 1.5% w/w, less than about 1% w/w) of the recombinant protein in the purified composition is high molecular weight species of the recombinant protein.

In some embodiments, the purified composition comprises at least about 85% w/w (e.g., at least about 90% w/w, at least about 95% w/w) of the recombinant protein in the composition prior to the loading.

In some embodiments, less than about 1% w/w of the recombinant protein in the purified composition is high molecular weight species of the recombinant protein; and the purified composition comprises at least about 90% w/w of the recombinant protein in the composition prior to the loading. In some embodiments, the loading density is about 100 g/L to about 600 g/L (e.g., about 150 g/L to about 600 g/L; about 200 g/L to about 600 g/L; about 250 g/L to about 600 g/L) of anion exchange material; less than about 1% w/w of the recombinant protein in the purified composition is high molecular weight species of the recombinant protein; and the purified composition comprises at least about 90% w/w of the recombinant protein in the composition prior to the loading.

In some embodiments, the recombinant protein is an antigen-binding protein. In some embodiments, the recombinant protein is an antibody. In some embodiments, the recombinant protein is a human antibody.

In some embodiments, the recombinant protein is an IgG1, IgG2, or IgG4 antibody. In some embodiments, the recombinant protein is a human IgG1, IgG2, or IgG4 antibody.

In some embodiments, the recombinant protein is an IgG1 antibody. In some embodiments, the recombinant protein is a human IgG1 antibody.

In some embodiments, the recombinant protein is an IgG2 antibody. In some embodiments, the recombinant protein is a human IgG2 antibody.

In some embodiments, the recombinant protein is an IgG4 antibody. In some embodiments, the recombinant protein is a human IgG4 antibody.

In some embodiments, the at least one impurity is selected from host cell proteins, nucleic acids, high molecular weight species of the recombinant protein, fragments of the recombinant protein, cell culture media components, and viral contaminants. In some embodiments, the at least one impurity is selected from high molecular weight species of the recombinant protein.

Also provided herein is a method for purifying a recombinant protein (e.g., an antigen-binding protein, such as, e.g., an antibody) from a composition comprising the recombinant protein and at least one impurity (e.g., a high molecular weight species of the recombinant protein), the method comprising:
loading the composition onto an anion exchange material comprising resin particles, wherein the resin particles comprise polymethacrylate and are functionalized with a primary amine ligand, at a loading density of about 250 g/L to about 600 g/L of anion exchange material, wherein:
   the composition has a pH of about 7.0 to about 8.0 and a conductivity of less than about 10 mS/cm (e.g., about 3 mS/cm to about 6 mS/cm); and
   the at least one impurity binds to the anion exchange material more strongly than the recombinant protein binds to the anion exchange material; and
collecting a purified composition comprising the recombinant protein.

In some embodiments, at least about 80% of the resin particles have a particle size of about 30 μm to about 60 μm. In some embodiments, the resin particles have an average particle size of about 40 μm to about 50 μm (such as, e.g., about 45 μm).

In some embodiments, the anion exchange material comprises resin particles, wherein the resin particles comprise a hydroxylated methacrylic polymer and are functionalized with a primary amine ligand. In some embodiments, the anion exchange material comprises resin particles, wherein the resin particles comprise a hydroxylated methacrylic polymer and are functionalized with a primary amine ligand, and further wherein at least about 80% of the resin particles have a particle size of about 30 μm to about 60 μm. In some embodiments, the anion exchange material comprises resin particles, wherein the resin particles comprise a hydroxylated methacrylic polymer and are functionalized with a primary amine ligand, and further wherein the resin particles have an average particle size of about 40 μm to about 50 μm (such as, e.g., about 45 μm).

In some embodiments, less than about 2.5% w/w of the recombinant protein in the purified composition is high molecular weight species of the recombinant protein. In some embodiments, the purified composition comprises at least about 85% w/w of the recombinant protein in the composition prior to the loading. In some embodiments, less than about 2.5% w/w of the recombinant protein in the purified composition is high molecular weight species of the recombinant protein, and the purified composition comprises at least about 85% w/w of the recombinant protein in the composition prior to the loading.

Also provided herein is a method for purifying a recombinant protein (e.g., an antigen-binding protein, such as, e.g., an antibody) from a composition comprising the recombinant protein and at least one impurity (e.g., a high molecular weight species of the recombinant protein), the method comprising:
loading the composition onto an anion exchange material comprising a primary amine ligand at a loading density of about 250 g/L to about 600 g/L of anion exchange material, wherein:
   the composition has a pH of about 7.0 to about 8.0 and a conductivity of about 3 mS/cm to about 6 mS/cm; and
   the at least one impurity binds to the anion exchange material more strongly than the recombinant protein binds to the anion exchange material; and collecting a purified composition comprising the recombinant protein, wherein:
   less than about 2.5% w/w of the recombinant protein in the purified composition is high molecular weight species of the recombinant protein; and/or
   the purified composition comprises at least about 85% w/w of the recombinant protein in the composition prior to the loading.

In some embodiments, the at least one impurity is selected from host cell proteins, nucleic acids, high molecular weight species of the recombinant protein, fragments of the recombinant protein, cell culture media components, and viral contaminants.

In some embodiments, the at least one impurity is selected from high molecular weight species of the recombinant protein.

In some embodiments, the anion exchange material comprises a polyamine ligand.

In some embodiments, the anion exchange material comprises a methacrylate-containing polymer base matrix.

In some embodiments, the anion exchange material comprises a methacrylate-containing polymer base matrix and a polyamine ligand.

In some embodiments, the anion exchange material comprises resin particles, wherein the resin particles comprise polymethacrylate and are functionalized with a primary amine ligand. In some embodiments, the anion exchange material comprises resin particles, wherein the resin particles comprise polymethacrylate and are functionalized with a primary amine ligand, and further wherein at least about 80% of the resin particles have a particle size of about 30 μm to about 60 μm. In some embodiments, the anion exchange material comprises resin particles, wherein the resin particles comprise polymethacrylate and are functionalized with a primary amine ligand, and further the resin particles have an average particle size of about 40 μm to about 50 μm (such as, e.g., about 45 μm).

In some embodiments, the anion exchange material comprises resin particles, wherein the resin particles comprise a hydroxylated methacrylic polymer and are functionalized with a primary amine ligand. In some embodiments, the anion exchange material comprises resin particles, wherein the resin particles comprise a hydroxylated methacrylic polymer and are functionalized with a primary amine ligand, and further wherein at least about 80% of the resin particles have a particle size of about 30 μm to about 60 μm. In some embodiments, the anion exchange material comprises resin particles, wherein the resin particles comprise a hydroxylated methacrylic polymer and are functionalized with a primary amine ligand, and further wherein the resin particles have an average particle size of about 40 μm to about 50 μm (such as, e.g., about 45 μm).

In some embodiments, less than about 2.5% w/w of the recombinant protein in the purified composition is high molecular weight species of the recombinant protein. In some embodiments, the purified composition comprises at least about 85% w/w of the recombinant protein in the composition prior to the loading. In some embodiments, less than about 2.5% w/w of the recombinant protein in the purified composition is high molecular weight species of the recombinant protein, and the purified composition comprises at least about 85% w/w of the recombinant protein in the composition prior to the loading.

Further provided herein is a method for purifying a recombinant protein (e.g., an antigen-binding protein, such as, e.g., an antibody) from a composition comprising the recombinant protein and at least one impurity (e.g., a high molecular weight species of the recombinant protein), the method comprising:
loading the composition onto an anion exchange material comprising a polyamine ligand at a loading density of about 250 g/L to about 600 g/L of anion exchange material, wherein:
   the composition has a pH of about 7.0 to about 8.0 and a conductivity of about 3 mS/cm to about 6 mS/cm; and
   the at least one impurity binds to the anion exchange material more strongly than the recombinant protein binds to the anion exchange material; and collecting a purified composition comprising the recombinant protein, wherein:
   less than about 2.5% w/w of the recombinant protein in the purified composition is high molecular weight species of the recombinant protein; and/or
   the purified composition comprises at least about 85% w/w of the recombinant protein in the composition prior to the loading.

In some embodiments, the at least one impurity is selected from host cell proteins, nucleic acids, high molecular weight species of the recombinant protein, fragments of the recombinant protein, cell culture media components, and viral contaminants.

In some embodiments, the at least one impurity is selected from high molecular weight species of the recombinant protein.

In some embodiments, the anion exchange material further comprises a methacrylate-containing polymer base matrix.

In some embodiments, less than about 2.5% w/w of the recombinant protein in the purified composition is high molecular weight species of the recombinant protein. In some embodiments, the purified composition comprises at least about 85% w/w of the recombinant protein in the composition prior to the loading. In some embodiments, less than about 2.5% w/w of the recombinant protein in the purified composition is high molecular weight species of the recombinant protein, and the purified composition comprises at least about 85% w/w of the recombinant protein in the composition prior to the loading.

Also provided herein is a method for purifying a recombinant protein (e.g., an antigen-binding protein, such as, e.g., an antibody) from a composition comprising the recombinant protein and at least one impurity (e.g., a high molecular weight species of the recombinant protein), the method comprising:
performing a low pH viral inactivation unit operation employing formic acid as an acid titrant;
loading the composition onto an anion exchange material comprising a primary amine ligand at a loading density of about 250 g/L to about 600 g/L of anion exchange material, wherein:
   the composition has a pH of about 7.0 to about 8.0 and a conductivity of less than 10 mS/cm (such as, e.g., about 3 mS/cm to about 6 mS/cm);
   the at least one impurity binds to the anion exchange material more strongly than the recombinant protein binds to the anion exchange material; and
   the low pH viral inactivation unit operation is performed one or more unit operations prior to the loading; and
collecting a purified composition comprising the recombinant protein.

In some embodiments, the at least one impurity is selected from host cell proteins, nucleic acids, high molecular weight species of the recombinant protein, fragments of the recombinant protein, cell culture media components, and viral contaminants.

In some embodiments, the at least one impurity is selected from high molecular weight species of the recombinant protein.

In some embodiments, the acid titrant is about 1M to about 2M formic acid. In some embodiments, the acid titrant is about 1M formic acid. In some embodiments, the acid titrant is about 2M formic acid.

In some embodiments, the anion exchange material comprises a polyamine ligand.

In some embodiments, the anion exchange material comprises a methacrylate-containing polymer base matrix.

In some embodiments, the anion exchange material comprises a methacrylate-containing polymer base matrix and a polyamine ligand.

In some embodiments, the anion exchange material comprises resin particles, wherein the resin particles comprise polymethacrylate and are functionalized with a primary amine ligand. In some embodiments, the anion exchange material comprises resin particles, wherein the resin particles comprise polymethacrylate and are functionalized with a primary amine ligand, and further wherein at least about 80% of the resin particles have a particle size of about 30 μm to about 60 μm. In some embodiments, the anion exchange material comprises resin particles, wherein the resin particles comprise polymethacrylate and are functionalized with a primary amine ligand, and further the resin particles have an average particle size of about 40 μm to about 50 μm (such as, e.g., about 45 μm).

In some embodiments, the anion exchange material comprises resin particles, wherein the resin particles comprise a hydroxylated methacrylic polymer and are functionalized with a primary amine ligand. In some embodiments, the anion exchange material comprises resin particles, wherein the resin particles comprise a hydroxylated methacrylic polymer and are functionalized with a primary amine ligand, and further wherein at least about 80% of the resin particles have a particle size of about 30 μm to about 60 μm. In some embodiments, the anion exchange material comprises resin particles, wherein the resin particles comprise a hydroxylated methacrylic polymer and are functionalized with a primary amine ligand, and further wherein the resin particles have an average particle size of about 40 μm to about 50 μm (such as, e.g., about 45 μm).

In some embodiments, less than about 2.5% w/w of the recombinant protein in the purified composition is high molecular weight species of the recombinant protein. In some embodiments, the purified composition comprises at least about 85% w/w of the recombinant protein in the composition prior to the loading. In some embodiments, less than about 2.5% w/w of the recombinant protein in the purified composition is high molecular weight species of the recombinant protein, and the purified composition comprises at least about 85% w/w of the recombinant protein in the composition prior to the loading.

Further provided herein is a method for purifying a recombinant protein (e.g., an antigen-binding protein, such as, e.g., an antibody) from a composition comprising the recombinant protein and at least one impurity selected from high molecular weight species of the recombinant protein, the method comprising:
performing a low pH viral inactivation unit operation employing about 1M to about 2M formic acid as an acid titrant;
loading the composition onto an anion exchange material comprising a polyamine ligand at a loading density of about 250 g/L to about 600 g/L of anion exchange material, wherein:
   the composition has a pH of about 7.0 to about 8.0 and a conductivity of less than 10 mS/cm (such as, e.g., about 3 mS/cm to about 6 mS/cm);
   the at least one impurity binds to the anion exchange material more strongly than the recombinant protein binds to the anion exchange material; and
   the low pH viral inactivation unit operation is performed one or more unit operations prior to the loading; and
collecting a purified composition comprising the recombinant protein.

In some embodiments, the acid titrant is about 1M formic acid. In some embodiments, the acid titrant is about 2M formic acid.

In some embodiments, the anion exchange material further comprises a methacrylate-containing polymer base matrix.

In some embodiments, less than about 2.5% w/w of the recombinant protein in the purified composition is high molecular weight species of the recombinant protein. In some embodiments, the purified composition comprises at least about 85% w/w of the recombinant protein in the composition prior to the loading. In some embodiments, less than about 2.5% w/w of the recombinant protein in the purified composition is high molecular weight species of the recombinant protein, and the purified composition comprises at least about 85% w/w of the recombinant protein in the composition prior to the loading.

Also provided herein is a method for purifying a recombinant protein (e.g., an antigen-binding protein, such as, e.g., an antibody) from a composition comprising the recombinant protein and at least one impurity (e.g., a high molecular weight species of the recombinant protein), the method comprising:
performing a low pH viral inactivation unit operation employing formic acid as an acid titrant;
loading the composition onto an anion exchange material comprising a primary amine ligand at a loading density of about 250 g/L to about 600 g/L of anion exchange material, wherein:
   the composition has a pH of about 7.0 to about 8.0 and a conductivity of about 3 mS/cm to about 6 mS/cm;
   the at least one impurity binds to the anion exchange material more strongly than the recombinant protein binds to the anion exchange material; and
   the low pH viral inactivation unit operation is performed one or more unit operations prior to the loading; and collecting a purified composition comprising the recombinant protein, wherein:
   less than about 2.5% w/w of the recombinant protein in the purified composition is high molecular weight species of the recombinant protein; and/or
   the purified composition comprises at least about 85% w/w of the recombinant protein in the composition prior to the loading.

In some embodiments, the anion exchange material comprises a polyamine ligand.

In some embodiments, the anion exchange material comprises a methacrylate-containing polymer base matrix.

In some embodiments, the anion exchange material comprises a methacrylate-containing polymer base matrix and a polyamine ligand.

In some embodiments, the anion exchange material comprises resin particles, wherein the resin particles comprise polymethacrylate and are functionalized with a primary amine ligand. In some embodiments, the anion exchange material comprises resin particles, wherein the resin particles comprise polymethacrylate and are functionalized with a primary amine ligand, and further wherein at least about 80% of the resin particles have a particle size of about 30 μm to about 60 μm. In some embodiments, the anion exchange material comprises resin particles, wherein the resin particles comprise polymethacrylate and are functionalized with a primary amine ligand, and further the resin particles have an average particle size of about 40 μm to about 50 μm (such as, e.g., about 45 μm).

In some embodiments, the anion exchange material comprises resin particles, wherein the resin particles comprise a hydroxylated methacrylic polymer and are functionalized with a primary amine ligand. In some embodiments, the anion exchange material comprises resin particles, wherein the resin particles comprise a hydroxylated methacrylic polymer and are functionalized with a primary amine ligand, and further wherein at least about 80% of the resin particles have a particle size of about 30 μm to about 60 μm. In some embodiments, the anion exchange material comprises resin particles, wherein the resin particles comprise a hydroxylated methacrylic polymer and are functionalized with a primary amine ligand, and further wherein the resin particles have an average particle size of about 40 μm to about 50 μm (such as, e.g., about 45 μm).

In some embodiments, less than about 2.5% w/w of the recombinant protein in the purified composition is high molecular weight species of the recombinant protein. In some embodiments, the purified composition comprises at least about 85% w/w of the recombinant protein in the composition prior to the loading. In some embodiments, less than about 2.5% w/w of the recombinant protein in the purified composition is high molecular weight species of the recombinant protein, and the purified composition comprises at least about 85% w/w of the recombinant protein in the composition prior to the loading.

### Host Cells

Cell lines (also referred to as “cells” or “host cells”) used in the present disclosure are genetically engineered to express a recombinant protein of commercial or scientific interest. Cells may be suitable for adherent, monolayer, and/or suspension culture, transfection, and expression of recombinant proteins, such as, e.g., antibodies. The cells can be used, for example, with batch, fed batch, and perfusion or continuous culture methods. Such cells are typically cell lines obtained or derived from mammals and are able to grow and survive when placed in either monolayer culture or suspension culture in medium containing appropriate nutrients and/or other factors, such as those described herein. Host cells are typically selected that can express and secrete proteins, or that can be molecularly engineered to express and secrete, large quantities of a particular protein, more particularly, a glycoprotein of interest, into the culture medium. The selection of an appropriate host cell for expressing a recombinant protein will depend upon various factors, such as desired expression levels, polypeptide modifications that are desirable or necessary for activity (such as glycosylation or phosphorylation), and ease of folding into a biologically active molecule. In some embodiments, the host cell producing the recombinant protein to be purified by a method provided herein is a mammalian host cell.

Cell lines are typically derived from a lineage arising from a primary culture that can be maintained in culture for an unlimited time. The cells can contain introduced, e.g., via transformation, transfection, infection, or injection, expression vectors (constructs), such as plasmids and the like, that harbor coding sequences, or portions thereof, encoding the proteins for expression and production in the culturing process. Such expression vectors contain the necessary elements for the transcription and translation of the inserted coding sequence. Methods which are well known to and practiced by those skilled in the art can be used to construct expression vectors containing sequences encoding the desired proteins and polypeptides, as well as the appropriate transcriptional and translational control elements. These methods include, but are not limited to, in vitro recombinant DNA techniques, synthetic techniques, and in vivo genetic recombination. Such techniques are described in J. Sambrook et al., 2012, Molecular Cloning, A Laboratory Manual, 4^{th} edition Cold Spring Harbor Press, Plainview, N.Y. or any of the previous editions; F. M. Ausubel et al., 2013, Current Protocols in Molecular Biology, John Wiley & Sons, New York, N.Y, or any of the previous editions; Kaufman, R.J., Large Scale Mammalian Cell Culture, 1990, all of which are incorporated herein for any purpose.

Suitable host cells include, but are not limited to, those that are commercially available, for example, from culture collections such as the DSMZ (Deutsche Sammlung von Mikroorganismen and Zellkulturen GmbH, Braunschweig, Germany) or the American Type Culture Collection (ATCC).

Example host cells include, but are not limited to, prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. In some embodiments, eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g., P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Vertebrate host cells are also suitable hosts for expressing recombinant proteins. Mammalian cell lines suitable as hosts for recombinant protein expression are well-known in the art and include, but are not limited to, immortalized cell lines available from the American Type Culture Collection (ATCC), including, but not limited to, Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. In some embodiments, the host cells are selected from CHO cells.

In some embodiments, the host cells are eukaryotic cells, such as, e.g., mammalian cells. The mammalian cells can be, for example, human or rodent or bovine cell lines or cell strains. Examples of such cells, cell lines, or cell strains include, but are not limited to, mouse myeloma (NSO)-cell lines, Chinese hamster ovary (CHO)-cell lines, FIT 1080, H9, HepG2, MCF7, MDBK Jurkat, NIH3T3, PC12, BF1K (baby hamster kidney cell), VERO, SP2/0, YB2/0, Y0, C127, L cell, COS, e.g., COS1 and COS7, QC1-3, HEK-293, VERO, PER.C6, HeLa, EB1, EB2, EB3, oncolytic, or hybridoma-cell lines. In some embodiments, the mammalian cells are CHO-cell lines. In some embodiments, the mammalian cells are CHO cells. In some embodiments, the mammalian cells are selected from CHO-K1 cells, CHO-K1 SV cells, DG44 CHO cells, DUXB11 CHO cells, CHOS cells, CHO GS knock-out cells, CHO FUT8 GS knock-out cells, CHOZN cells, and CHO derived cells. In some embodiments, a CHO GS knock-out cell (such as, e.g., a GSKO cell) is, for example, a CHO-K1 SV GS knockout cell. Additionally, the CHO FUT8 knockout cell is, for example, the Potelligent® CHOK1 SV (Lonza, Inc.). In some embodiments, the eukaryotic cells can also be avian cells, cell lines, or cell strains, such as, e.g., EBx® cells, EB14, EB24, EB26, EB66, or EBv13.

CHO cells, including CHOK1 cells (ATCC CCL61), are widely used to produce complex recombinant proteins. In some embodiments, the dihydrofolate reductase (DHFR)-deficient mutant cell lines (Urlaub et al., 1980, Proc Natl Acad Sci USA 77: 4216-4220), DXB11 and DG-44, are desirable CHO host cell lines because the efficient DHFR selectable and amplifiable gene expression system allows high level recombinant protein expression in these cell lines (Kaufman R. J., 1990, Meth Enzymol 185:537-566). Also included are the glutamine synthase (GS)-knockout CHOK1SV cell lines, making use of glutamine synthetase (GS)-based methionine sulfoximine (MSX) selection. Other suitable CHO host cells include, but are not limited to, the following (ECACC accession numbers in parenthesis): CHO (85050302); CHO (PROTEIN FREE) (00102307); CHO-K1 (85051005); CHO-K1/SF (93061607); CHO/dhFr-(94060607); CHO/dhFr-AC-free (05011002); and RR-CHOK1 (92052129).

Large-scale production of proteins for commercial applications may be carried out in suspension culture. Therefore, mammalian host cells used to generate the recombinant mammalian cells described herein can, but need not be, adapted to growth in suspension culture. A variety of host cells adapted to growth in suspension culture are known, including mouse myeloma NSO cells and CLIO cells from CFIO-S, DG44, and DXB11 cell lines. Other suitable cell lines include, but are not limited to, mouse myeloma SP2/0 cells, baby hamster kidney BF1K-21 cells, human PER.C6® cells, human embryonic kidney F1EK-293 cells, and cell lines derived or engineered from any of the cell lines disclosed herein.

In some embodiments, the eukaryotic cells are selected from lower eukaryotic cells, such as, e.g., yeast cells (e.g., Pichia genus (e.g., Pichia pastoris, Pichia methanolica, Pichia kluyveri, and Pichia angusta), Komagataella genus (e.g., Komagataella pastoris, Komagataella pseudopastoris, or Komagataella phaffii), cells of the Saccharomyces genus (e.g., Saccharomyces cerevisae, Saccharomyces kluyveri, Saccharomyces uvarum), cells of the Kluyveromyces genus (e.g., Kluyveromyces lactis, Kluyveromyces marxianus), cells of the Candida genus (e.g., Candida utilis, Candida cacaoi, Candida boidinii), cells of the Geotrichum genus (e.g., Geotrichum fermentans), Hαη-senula polymorpha, Yarrowia lipolytica, or Schizosaccharomyces pombe. In some embodiments, the eukaryotic cells are selected from Pichia pastoris strains. Non-limiting examples of Pichia pastoris strains include X33, GS115, KM71, KM71H, and CBS7435.

In some embodiments, the eukaryotic cells are selected from fungal cells (e.g., cells of Aspergillus (such as, e.g., A. niger, A. fumigatus, A. orzyae, A. nidula), Acremonium (such as, e.g., A. thermophilum), Chaetomium (such as, e.g., C. thermophilum), Chrysosporium (such as, e.g., C. thermophile), Cordyceps (such as, e.g., C. militaris), Corynascus, Ctenomyces, Fusarium (such as, e.g., F. oxysporum), Glomerella (such as, e.g., G. graminicola), Hypocrea (such as, e.g., H. jecorina), Magnaporthe (such as, e.g., M. orzyae), Myceliophthora (such as, e.g., M. thermophile), Nectria (such as, e.g., N. heamatococca), Neurospora (such as, e.g., N. crassa), Penicillium, Sporotrichum (such as, e.g., S. thermophile), Thielavia (such as, e.g., T. terrestris, T. heterothallica), Trichoderma (such as, e.g., T. reesei), or Verticillium (such as, e.g., V. dahlia)).

In some embodiments, the eukaryotic cells are selected from insect cells (such as, e.g., Sf9, Mimic™ Sf9, Sf21, High Five™ (BT1-TN-5B1-4), or BT1-Ea88 cells), algae cells (such as, e.g., of the genus Amphora, Bacillariophyceae, Dunaliella, Chlorella, Chlamydomonas, Cyanophyta (cyanobacteria), Nannochloropsis, Spirulina, or Ochromonas), and plant cells (such as, e.g., cells from monocotyledonous plants (such as, e.g., maize, rice, wheat, or Setaria), or cells from a dicotyledonous plants (such as, e.g., cassava, potato, soybean, tomato, tobacco, alfalfa, Physcomitrella patens or Arabidopsis)).

To generate host cell lines (e.g., mammalian cell lines) engineered to express a recombinant protein of interest, one or more nucleic acids encoding the recombinant protein (or components thereof in the case of multi-chain proteins) is initially inserted into one or more expression vectors. Nucleic acid control sequences useful in expression vectors for expression in mammalian cells include promoters, enhancers, and termination and polyadenylation signals. A secretory signal peptide sequence can also, optionally, be encoded by the expression vector, operably linked to the coding sequence of interest, so that the expressed protein can be secreted by the recombinant host cell, for more facile isolation of the recombinant protein from the cell, if desired. Vectors may also include one or more selectable marker genes to facilitate selection of host cells into which the vectors have been introduced. In some embodiments, vectors are used that employ protein-fragment complementation assays using protein reporters, such as dihydrofolate reductase (see, for example, U.S. Pat. No. 6,270,964). Suitable mammalian expression vectors are known in the art and are also commercially available.

Typically, vectors used in any of the host cells will contain sequences for plasmid maintenance and for cloning and expression of exogenous nucleotide sequences. Such sequences will typically include one or more of the following nucleotide sequences: a promoter, one or more enhancer sequences, an origin of replication, transcriptional and translational control sequences, a transcriptional termination sequence, a complete intron sequence containing a donor and acceptor splice site, a native or heterologous signal peptide sequence (leader sequence or signal peptide) for polypeptide secretion, a ribosome binding site, a polyadenylation sequence, a polylinker region for inserting the polynucleotide encoding the polypeptide to be expressed, and a selectable marker element. Vectors may be constructed from a starting vector such as a commercially available vector, and additional elements may be individually obtained and ligated into the vector.

### Culture Methods

Various culture methods may be used to produce a recombinant protein of interest, including, but not limited to, batch culture, fed-batch culture, and perfusion culture.

Batch culture is a discontinuous method where cells are grown in a fixed volume of culture media for a short period of time followed by a full harvest. Cultures grown using the batch method experience an increase in cell density until a maximum cell density is reached, followed by a decline in viable cell density as the media components are consumed and levels of metabolic by-products (such as lactate and ammonia) accumulate. Harvest typically occurs at the point when the maximum cell density is achieved (e.g., 5×10⁶ cells/mL or greater, depending on media formulation, cell line, etc.). The batch process is the simplest culture method; however, viable cell density is limited by nutrient availability and once the cells are at maximum density, the culture declines and production decreases. There is no ability to extend a production phase in batch culture because the accumulation of waste products and nutrient depletion rapidly lead to culture decline, typically around 3 to 7 days.

Fed-batch culture improves on the batch process by providing bolus or continuous media feeds to replenish those media components that have been consumed. Since fed-batch cultures receive additional nutrients throughout the run, they have the potential to achieve higher cell densities (>10 to 30×10⁶ cells/mL, depending on media formulation, cell line, etc.) and increased product titers, when compared to the batch method. Unlike the batch process, a biphasic culture can be created and sustained by manipulating feeding strategies and media formulations to distinguish the period of cell proliferation to achieve a desired cell density (the growth phase) from the period of suspended or slow cell growth (the production phase). As such, fed-batch cultures have the potential to achieve higher product titers compared to batch cultures. Typically, a batch method is used during the growth phase and a fed-batch method used during the production phase, but a fed-batch feeding strategy can be used throughout the entire process. However, unlike the batch process, bioreactor volume is a limiting factor which limits the amount of feed. Also, as with the batch method, metabolic by-product accumulation will lead to culture decline, which limits the duration of the production phase, often around 10 to 21 days. Fed-batch cultures are discontinuous, and harvest typically occurs when metabolic by-product levels or culture viability reach predetermined levels. When compared to a batch culture, in which no feeding occurs, a fed-batch culture can produce greater amounts of recombinant protein. (See, e.g., U.S. Pat. No. 5,672,502.)

Perfusion methods offer potential improvements over the batch and fed-batch methods by adding fresh media and simultaneously removing spent media during culture. Typical perfusion cultures begin with a batch culture start-up lasting for a day or two followed by continuous, step-wise, and/or intermittent addition of fresh feed media to the culture and simultaneous removal of spent media with the retention of cells and additional high molecular weight compounds such as proteins (based on the filter molecular weight cutoff) throughout the growth and production phases of the culture. Various methods, such as sedimentation, centrifugation, or filtration, can be used to remove spent media, while maintaining cell density. Non-limiting example filtration methods include tangential flow filtration (TFF), such as recirculating flow filtration and alternating tangential flow (ATF) filtration. Alternating tangential flow is maintained by pumping medium through hollow-fiber filter modules. See, e.g., U.S. Pat. No. 6,544,424; Furey, 2002, Gen. Eng. News. 22 (7):62-63.

Perfusion can be continuous, stepwise, intermittent, or a combination of any or all of any of these. Perfusion rates can be less than a working volume to many working volumes per day. The cells are retained in the culture, and the spent medium that is removed is substantially free of cells or has significantly fewer cells than the culture. Recombinant proteins expressed by the cell culture can also be retained in the culture.

Typical large scale commercial cell culture strategies strive to reach high cell densities, 40-90(+)×10⁶ cells/mL, such as, e.g., about 40×10⁶ cells/mL or about 50×10⁶ cells/mL, where almost a third to over one-half of the reactor volume is biomass. With perfusion culture, extreme cell densities of >1×10⁸ cells/mL have been achieved. A potential advantage of the perfusion process is that the production culture can be maintained for longer periods than batch or fed-batch culture methods. However, increased media preparation, use, storage, and disposal are necessary to support a long-term perfusion culture, particularly for a culture with high cell density, which also needs even more nutrients. In addition, higher cell densities can cause problems during production, such as, e.g., maintaining dissolved oxygen levels and problems with increased gassing, including supplying more oxygen and removing more carbon dioxide, which could result in more foaming and the need for alterations to antifoam strategies; as well as during harvest and downstream processing where the efforts required to remove the excessive cell material can result in loss of product, negating the benefit of increased titer due to increased cell mass.

Suitable culture conditions, including temperature, dissolved oxygen content, agitation rate, and the like, for mammalian cells are known in the art and may vary by the phase or stage of the cell culture. In some embodiments, the methods disclosed herein further comprise taking samples during the cell culture processes, evaluating the samples to quantitatively and/or qualitatively monitor characteristics of the recombinant protein and/or the cell culture process. In some embodiments, the samples are quantitatively and/or qualitatively monitored using process analytical techniques. For examples, dissolved oxygen levels may be monitored during the cell culture processes using methods known in the art, such as, e.g., a basic chemical analysis method (titration method), an electrochemical analysis method (diaphragm electrode method), and a photochemical analysis method (fluorescence method).

During recombinant protein production, it is desirable to have a controlled system where cells are grown for a desired time or to a desired density and then the physiological state of the cells is switched to a growth-limited or arrested, high productivity state where the cells use energy and substrates to produce the recombinant protein in favor of increasing cell density. For commercial scale cell culture and the manufacture of biological therapeutics, the ability to limit or arrest cell growth and to maintain the cells in a growth-limited or arrested state during the production phase is very desirable. Such methods include, for example, temperature shifts, use of chemical inducers of protein production, nutrient limitation or starvation, and cell cycle inhibitors, either alone or in combination. Illustratively, a typical cell culture undergoes a growth phase, a period of exponential growth where cell density is increased. During the growth phase, cells are cultured in a cell culture medium containing the necessary nutrients and additives under conditions (generally at about a temperature of 25°−40° C., in a humidified, controlled atmosphere) such that optimal growth is achieved for the particular cell line. Cells are typically maintained in the growth phase for a period of between one and eight days, e.g., between three to seven days, e.g., seven days. The length of the growth phase for a particular cell line can be determined by a person of ordinary skill in the art and will generally be the period of time sufficient to allow the particular cells to reproduce to a viable cell density within a range of about 20%-80% of the maximal possible viable cell density if the culture was maintained under the growth conditions. The growth phase is followed by a transition phase when exponential cell growth is slowing and protein production starts to increase. This marks the start of the stationary phase, a production phase, where cell density typically levels off and product titer increases. During the production phase, the medium is generally supplemented to support continued recombinant protein production.

In certain embodiments, the culture conditions used to produce a recombinant protein may be adjusted to facilitate the transition from the growth phase of the cell culture to the production phase. For instance, a growth phase of the cell culture may occur at a higher temperature than a production phase of the cell culture. In some embodiments, a growth phase may occur at a first temperature from about 35° C. to about 38° C., and a production phase may occur at a second temperature from about 29° C. to about 37° C., optionally from about 30° C. to about 36° C. or from about 30° C. to about 34° C. In one embodiment, a shift in temperature from about 35° C. to about 37° C. to a temperature of about 31° C. to about 33° C. may be employed to facilitate the transition from the growth phase of the culture to the production phase. Chemical inducers of protein production, such as, for example, caffeine, butyrate, and hexamethylene bisacetamide (HMBA), may be added at the same time as, before, and/or after a temperature shift, or in place of a temperature shift. If inducers are added after a temperature shift, they can be added from one hour to five days after the temperature shift, optionally from one to two days after the temperature shift.

Additionally, any cell culture media capable of supporting growth of the appropriate host cell in culture can be used. Typically, the cell culture medium contains a buffer, salts, energy source, amino acids, vitamins, and trace essential elements. Cell culture media, which may be further supplemented with other components to maximize cell growth, cell viability, and/or recombinant protein production in a particular cultured host cell, are commercially available and include RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series, among others, which can be obtained from the American Type Culture Collection or SAFC Biosciences, as well as other vendors. Cell culture media can be serum-free, protein-free, growth factor-free, and/or peptone-free media. Cell culture media may also be enriched by the addition of nutrients or other supplements, which may be used at greater than usual, recommended concentrations. In certain embodiments, the culture medium used in the production of a recombinant protein to be purified by a method provided herein is a chemically defined medium, which refers to a cell culture medium in which all of the components have known chemical structures and concentrations. Chemically defined media are typically serum-free and do not contain hydrolysates or animal-derived components.

Various media formulations can be used during the life of the culture, for example, to facilitate the transition from one stage (e.g., the growth stage or phase) to another (e.g., the production stage or phase) and/or to optimize conditions during cell culture (e.g., concentrated media provided during a perfusion culture). A growth medium formulation can be used to promote cell growth and minimize protein expression. A production medium formulation can be used to promote production of the recombinant protein of interest and maintenance of the cells, with minimal new cell growth. A feed medium is typically a cell culture medium containing more concentrated components such as nutrients and amino acids, which are consumed during the course of the production phase of the cell culture. A feed medium may be used to supplement and maintain an active culture, particularly a culture operated in fed batch, semi-perfusion, or perfusion mode. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount.

In some embodiments, the mammalian cell used to produce a recombinant protein is cultured for a defined period of time during which the recombinant protein is expressed and secreted by the mammalian cell. This period of time (i.e., the duration of the production phase of the cell culture) is at least 3 days, at least 7 days, at least 10 days, or at least 15 days. In certain embodiments, the duration of the production phase of the cell culture is about 7 days to about 28 days, about 10 days to about 30 days, about 7 days to about 14 days, about 10 days to about 18 days, about 3 days to about 15 days, about 5 days to about 8 days, about 12 days to about 15 days, about 12 days to about 18 days, or about 15 days to about 21 days. In some embodiments, the duration of the production phase of the cell culture is 7 days, 8 days, 9 days, 12 days, 15 days, 18 days, or 21 days.

In some embodiments, the biomanufacturing process to produce a recombinant protein comprises a production phase with a viable cell density of at least 100×10⁵ cells/mL, for example, between about 100×10⁵ cells/mL and about 10×10⁷ cells/mL, between about 250×10⁵ cells/mL and about 900×10⁵ cells/mL, between about 300×10⁵ cells/mL and 800×10⁵ cells/mL, or between about 450×10⁵ cells/mL and 650×10⁵ cells/mL. Cell density may be measured using a hemacytometer, a Coulter counter, or an automated cell analyzer (e.g., Cedex automated cell counter). Viable cell density may be determined by staining a culture sample with Trypan blue, which is taken up only by dead cells. Viable cell density is then determined by counting the total number of cells, dividing the number of stained cells by the total number of cells, and taking the reciprocal.

In some embodiments, the upstream biomanufacturing process that produces a recombinant protein to be purified by a method provided herein comprises a production phase with a packed cell volume less than or equal to 35%. In some embodiments, the packed cell volume is less than or equal to 30%.

Critical attributes and performance indicators of the recombinant protein of interest can be measured to better inform decisions regarding performance of each step during manufacture. These critical attributes and performance indicators can be monitored real-time, near real-time, and/or off-line. Critical parameters that can be measured during cell culture may include cell culture media components that are consumed (such as, e.g., glucose), levels of metabolic by-products (such as, e.g., lactate and ammonia) that accumulate, as well as those related to cell maintenance and survival, such as dissolved oxygen content. Additionally, critical attributes such as specific productivity, viable cell density, packed cell volume, pH, osmolality, aggregation, percent yield, and titer may be monitored during appropriated stages in the manufacturing process. Monitoring and measurements can be performed using known techniques and commercially available equipment.

### Bioreactors

In some embodiments, the growth and/or production phase of an upstream process used to produce a recombinant protein is conducted within a bioreactor. Conditions within the bioreactor to support cell culture. Suitable culture conditions for mammalian cells are known in the art, as described above. A bioreactor “run” typically comprises the steps of inoculating a prepared bioreactor with a seed culture, subjecting the cells to one or more growth phase and/or production phases until one or more predetermined parameters are met (e.g., time, viable cell density, packed cell volume) and then harvesting the contents of the bioreactor.

In some embodiments, one or more bioreactor(s) used to produce a recombinant protein is a stainless-steel bioreactor, such as, e.g., a built-in-place large-scale stainless-steel bioreactor capable of operating at volumes of about 2,000 liters to about 50,000 liters (e.g., about 2,000 liters to about 20,000 liters) or more.

In some embodiments, one or more bioreactor(s) to produce a recombinant protein is a single-use bioreactor. Single-use technology minimizes the infrastructure requirements associated with traditional cell culture, such as steel/glass commercial-scale vessels and associated machinery. Single-use bioreactors provide flexibility to the manufacturing process, and site assembly, reconfiguration, sterilization, and validation for single-use bioreactors may be faster, easier, and less costly than traditional built-in-place stainless steel cell culture plants. Single-use bioreactors comprise disposable, plastic sterile bags supported by a non-disposable support structure. The culture is agitated by a stirrer within the bag or by rocking, air and oxygen spargers are also supplied as well as sensors to measure and adjust various parameters of the culture, such as pH, temperature, oxygen, cell density, and the like. Single-use bioreactors are commercially available, for example, Bio STR®, Sartorius, Gattingen Germany; MOBIUS®, Millipore, Burlington, MA; XCELLEREX®, Cytiva, Marlborough, MA.

Bioreactor volume is divided into the working volume space and the headspace. The working volume of the bioreactor refers to the volume within the bioreactor in which the cell culture is operated, typically expressed as a percentage of the bioreactor volume. In some embodiments, the working volume of the bioreactor is at least about 70% of the bioreactor volume. In some embodiments, the working volume of the bioreactor is at least about 70% to about 100% of the bioreactor volume. In some embodiments, the working volume of the bioreactor is at least about 75% of the bioreactor volume. In some embodiments, the working volume of the bioreactor is at least about 80% of the bioreactor volume. In some embodiments, the working volume of the bioreactor is at least about 85% of the bioreactor volume. In some embodiments, the working volume of the bioreactor is at least about 90% of the bioreactor volume. In some embodiments, the working volume of the bioreactor is at least about 91% of the bioreactor volume. In some embodiments, the working volume of the bioreactor is at least about 92% of the bioreactor volume. In some embodiments, the working volume of the bioreactor is at least about 93% of the bioreactor volume. In some embodiments, the working volume of the bioreactor is at least about 94% of the bioreactor volume. In some embodiments, the working volume of the bioreactor is at least about 95% of the bioreactor volume. In some embodiments, the working volume of the bioreactor is at least about 96% of the bioreactor volume. In some embodiments, the working volume of the bioreactor is at least about 97% of the bioreactor volume. In some embodiments, the working volume of the bioreactor is at least about 98% of the bioreactor volume. In some embodiments, the working volume of the bioreactor is at least about 99% of the bioreactor volume. In some embodiments, the working volume of the bioreactor is about 100% of the bioreactor volume.

### Additional Harvesting and Purification Processes

The expressed recombinant proteins may be secreted into the culture medium from which they can be recovered and/or collected. Some biomanufacturing processes that incorporate an anion exchange chromatography operation of the present disclosure may also include a harvesting operation. A harvesting operation fully or partially clarifies and/or purifies the target protein away from at least one impurity with which it is found in the cell culture fluid, such as remaining cell culture media, cells, cell debris, or media components, and/or other product-and/or process-related impurities.

Methods for harvesting recombinant proteins from suspension cell cultures are known in the art and include, but are not limited to, acid precipitation, accelerated sedimentation such as flocculation, separation using gravity, centrifugation, acoustic wave separation, filtration, including membrane filtration, ultrafilters, microfilters, tangential flow, alternating tangential flow, depth filters, and alluvial filtration filters.

The harvested cell culture fluid (HCCF) can be stored in surge tanks, holding tanks, bags, or other containers that are adapted to provide feed to a chromatography column skid and are appropriate for the infrastructure and/or process requirements.

Harvest operations may be combined with additional harvest strategies, including centrifugation, such as disk-stack centrifugation or continuous solid discharge centrifugation; filtration, including tangential flow filtration, microfiltration, ultrafiltration, and depth filtration; precipitation/sedimentation methods, such as flocculation; and chromatography media-based separations.

Beyond an anion exchange chromatography operation using an anion exchange chromatography material comprising a primary amine ligand, the present disclosure encompasses methods involving all known purification technologies, such as, e.g., protein A purification of immunoglobulin and immunoglobulin-like biologics, as well as chromatography-based separations and polishing steps that include column and alternative modes of chromatographic separations by ion exchange chromatography (IEX), including anion exchange chromatography (AEX) and/or cation exchange chromatography (CEX), hydrophobic interaction chromatography (HIC), mixed modal or multimodal chromatography (MM), hydroxyapatite chromatography (HA), reverse-phase chromatography, size exclusion chromatography (SEC), gel filtration, or any other known form of chromatographic separation of biological and/or biochemical substances.

In some embodiments, recombinant protein recovered from host cells or cell culture medium may be further purified or partially purified to remove cell culture media components, host cell proteins, or nucleic acids, or other process- or product-related impurities by one or more unit operations. One of ordinary skill in the art can select the appropriate unit operation(s) for further purification of a recombinant protein based on the characteristics of the recombinant protein to be purified, the characteristics of host cell from which the recombinant protein is expressed, and the composition of the culture medium in which the host cells were grown. Illustratively, in some embodiments, the recombinant protein is purified from the harvest permeate by one or more of flocculation, precipitation, centrifugation, depth filtration, affinity chromatography, size exclusion chromatography, ion exchange chromatography, mixed mode anion exchange chromatography, hydrophobic interaction chromatography, or hydroxyapatite chromatography.

A capture unit operation may include capture chromatography that makes use of resins and/or membranes containing agents that will bind to the recombinant protein of interest, for example, affinity chromatography, size exclusion chromatography, ion exchange chromatography, hydrophobic interaction chromatography (HIC), immobilized metal affinity chromatography (IMAC), and the like. Such chromatographic materials are known in the art and are commercially available. For instance, if the recombinant protein is an antibody or contains components derived from an antibody (e.g., a Fc domain), affinity chromatography using ligands such as Protein A, Protein G, Protein A/G, or Protein L may be employed as a capture chromatography unit operation to further purify the recombinant protein. In other embodiments, the recombinant protein of interest may comprise a polyhistidine tag at its amino or carboxyl terminus and subsequently purified using IMAC. Recombinant proteins can be engineered to include other purification tags, such as a FLAG® tag or c-myc epitope and subsequently purified by affinity chromatography using a specific antibody directed to such tag or epitope.

Unit operations directed towards inactivating, reducing, and/or eliminating viral contaminants may include processes that mitigate viral risk by manipulating the environment and/or through use of filtration. Viral mitigation measures are critical to ensure the safety of protein therapeutics and may be performed one or more times throughout the downstream purification. Viral contaminants can arise from a variety of sources, including use of reagents of animal origin, adventitious viral contaminants in host cell lines, or system failures at GMP manufacturing sites. Viruses are classified as enveloped and non-enveloped viruses. With enveloped viruses, the envelope allows the virus to identify, bind, enter, and infect target host cells. As such, enveloped viruses are susceptible to inactivation methods. Various methods can be employed for virus inactivation, including heat inactivation/pasteurization, UV and gamma ray irradiation, use of high intensity broad spectrum white light, addition of chemical inactivating agents, surfactants, and solvent/detergent treatments. Surfactants, such as detergents, solubilize membranes and can be very effective in specifically inactivating enveloped viruses. Additional unit operations to inactivate, reduce, and/or eliminate viral contaminants may include filtration processes and/or adjusting solution conditions. One method for achieving viral inactivation is incubation at low pH (e.g., pH<4). A low pH viral inactivation operation can be followed with a neutralization unit operation that readjusts the viral inactivated solution to a pH more compatible with the requirements of the subsequent unit operations. A low pH viral inactivation operation may also be followed by filtration, such as depth filtration, to remove any resulting turbidity or precipitation. Adjusting the temperature or chemical composition (e.g., use of detergents) can also be used to achieve viral inactivation. Viral filtration can be performed using micro- or nano-filters, such as those available from Asahi Kasei (Plavona®) and EMD Millipore (VPro®).

Non-enveloped viruses are less susceptible to inactivation methods that preserve product stability. Accordingly, non-enveloped viruses are typically removed by filtration methods. An example process is described in WO2020/159838. Viral filtration can be performed using micro-or nano-filters, such as those available from PLAVONA® (Asahi Kasei, Chicago, IL), VIROSART® (Sartorius, Goettingen, Germany), VIRESOLVE® Pro (MilliporeSigma, Burlington, MA), Pegasus™ Prime (Pall Biotech, Port Washington, NY), and CUNO Zeta Plus VR (3M, St. Paul, Mn).

Viral filtration may occur at one or more steps in the downstream operations of a biomanufacturing process. Typically, viral inactivation follows an affinity chromatography unit operation and viral filtration precedes or follows an ultrafiltration/diafiltration (UF/DF) operation but may also take place following UF/DF.

In all chromatography processes, multiple filters can be used to the capacity that holders, skids, or the physical set up of the ultrafiltration/diafiltration (UF/DF) system will allow or are needed to achieve the desired objectives of a production process.

A polishing unit operation may make use of various chromatographic methods for the purification of the protein of interest and clearance of contaminants and impurities. The polishing chromatography unit operation may make use of resins and/or membranes containing agents that can be used in either a “flow-through mode,” in which the protein of interest is contained in the eluent and the contaminants and impurities are bound to the chromatographic medium, or “bind-and-elute mode,” in which the protein of interest is bound to the chromatographic medium and eluted after the contaminants and impurities have flowed through or been washed off the chromatographic medium. Examples of such polishing chromatography methods include, but are not limited to, ion exchange chromatography (IEX), such as cation exchange chromatography (CEX); hydrophobic interaction chromatography (HIC); mixed modal or multimodal chromatography (MM), hydroxyapatite chromatography (HA); reverse phase chromatography, and size-exclusion chromatography (e.g., gel filtration).

Purified recombinant protein may be formulated, i.e., buffer exchanged, sterilized, bulk-packaged, and/or packaged for a final user. Illustratively, product concentration and buffer exchange of the recombinant protein of interest into a desired formulation buffer for bulk storage of the drug substance or drug product can be accomplished by ultrafiltration and/or diafiltration. Suitable formulations for pharmaceutical compositions include those described in Remington's Pharmaceutical Sciences, 18th ed. 1995, Mack Publishing Company, Easton, PA.

A UF/DF operation may take place at one or more stages in a downstream process. Typically, a UF/DF operation is performed prior to bulk storage of the drug substance. Instead of storage, unit operations related to drug product fill/finish can also immediately follow a UF/DF operation. One or more stability-enhancing excipients may optionally be added directly to the UF/DF retentate feed tank containing the formulated purified protein resulting in formulated drug substance or added to the UF/DF eluate pool. An example UF/DF process is described in WO 2020/159838. Filters for use in a UF/DF operation are well-known in the art and are commercially available from many sources. There are many types of materials available, such as regenerated cellulose, Pellicon (MilliporeSigma, Danvers, MA), stabilized cellulose, Sartocono Slice, Sartocono ECO Hydrosarto (Sartorius, Goettingen, Germany), and polyethersulfone (PES) membrane, Omega (Pall Corporation, Port Washington, NY).

### Recombinant Proteins

Any type of recombinant protein, including proteins containing single polypeptide chains or multiple polypeptide chains, can be purified according to the methods of the present disclosure. Such recombinant proteins include, but are not limited to, secreted proteins, non-secreted proteins, intracellular proteins, or membrane-bound proteins. Illustratively, recombinant proteins can include, but are not limited to, cytokines, growth factors, hormones, muteins, fusion proteins, antibodies, antibody fragments, peptibodies, T-cell engaging molecules, and multi-specific antigen binding proteins. In some embodiments, the recombinant protein is a fusion protein.

In other embodiments, the recombinant protein to be purified according to a method of the present disclosure is an antigen-binding protein. Antigen-binding proteins include, but are not limited to, antibodies, peptibodies, antibody derivatives, antibody analogs, fusion proteins (including, e.g., single-chain variable fragments (scFvs), double-chain (divalent) scFvs, and IgGscFv (see, e.g., Orcutt et al., 2010, Protein Eng Des Sel 23:221-228)), hetero-IgGs (see, e.g., Liu et al., 2015, J Biol Chem 290:7535-7562), muteins, and XmAb® (Xencor, Inc., Monrovia, CA). Additional antigen-binding proteins include, but are not limited to, bispecific T cell engagers (BiTE®), bispecific T cell engagers having extensions, such as, e.g., half-life extensions, such as, e.g., HLE BiTE molecules, HeteroIg BITE molecules, and others, chimeric antigen receptors (CARs, CAR Ts), and T cell receptors (TCRs).

In some embodiments, the antigen-binding protein binds to one of more of the following, alone or in any combination: CD proteins including, but not limited to, CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including, but not limited to, insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP; viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, DNase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCGβ, hepatitis-C virus, mesothelin dsFv[PE38] conjugate, Legionella pneumophila (lly), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein IIb/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In other embodiments, the recombinant protein to be purified according to a method of the present disclosure is an antibody. In some embodiments, the antibody is a human antibody.

In some embodiments, the antibody is selected from abrilumab, brazikumab, brodalumab, crizanlizumab, denosumab, eculizumab, erenumab, evolocumab, fremanezumab, meplazumab, nemolizumab, ontamalimab, panitumumab, prezalumab, ravulizumab, rilotumumab, romosozumab, satralizumab, tafolecimab, tanezumab, tezepelumab, tremelimumab, utomilumab, and volagidemab. In some embodiments, the antibody is selected from denosumab, erenumab, evolocumab, panitumumab, romosozumab, and tezepelumab. In some embodiments, the antibody is denosumab. In some embodiments, the antibody is erenumab. In some embodiments, the antibody is evolocumab. In some embodiments, the antibody is panitumumab. In some embodiments, the antibody is romosozumab. In some embodiments, the antibody is tezepelumab.

In some embodiments, the antibody is an IgG1, IgG2, or IgG4 antibody. In some embodiments, the antibody is a human IgG1, IgG2, or IgG4 antibody.

In some embodiments, the antibody is an IgG1 antibody. In some embodiments, the antibody is a human IgG1 antibody.

In some embodiments, the antibody is an IgG2 antibody. In some embodiments, the antibody is a human IgG2 antibody.

In some embodiments, the antibody is an IgG4 antibody. In some embodiments, the antibody is a human IgG4 antibody.

### EXAMPLES

In order that the present disclosure may be more fully understood, the following examples are set forth. It should be understood that these examples are for illustrative purposes only and are not to be construed as limiting this disclosure in any manner.

### Example 1: AEX Chromatography Step Using TOYOPEARL® NH2-750F

An AEX resin consisting of TOYOPEARL® NH2-750F (Tosoh Bioscience) was used to further polish compositions comprising one of two recombinant monoclonal antibodies, mAb1 or mAb2, following affinity chromatography, low pH viral inactivation, and CEX chromatography. TOYOPEARL® NH2-750F is composed of polymethacrylate beads that have been functionalized with proprietary primary amine (NH₂) strong anion exchange groups and is commercially available in a 45 μm particle size (F-grade). The operating pH for the AEX step was between 7.0 and 8.0, and the operating conductivity was less than 10 mS/cm. The AEX column was loaded at a loading density between 250 g/L-resin and 600 g/L-resin.

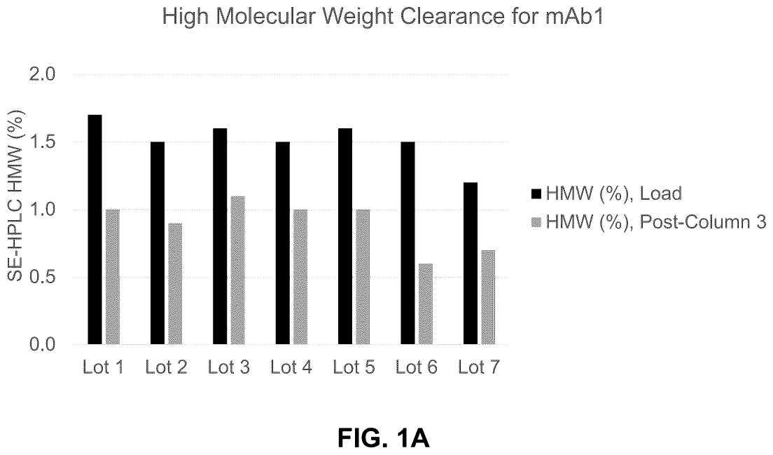

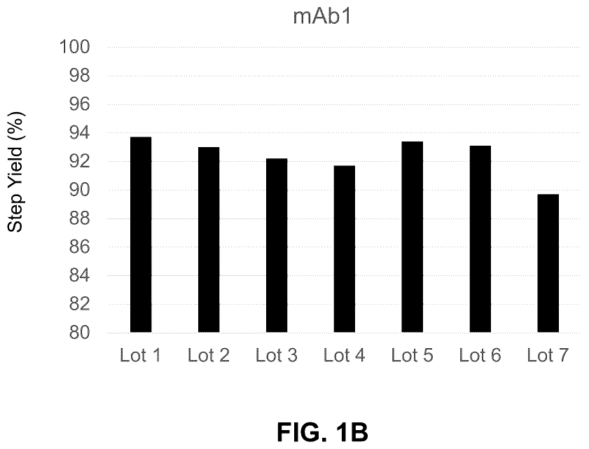

FIG. 1A shows the removal capabilities of this AEX step for overall HMW species of mAb1 by comparing HMW levels (as assessed by SE-HPLC) in the load and pool across seven pilot-scale lots. The observed HMW reduction of at least 0.5% across all three lots demonstrates the robustness of the AEX step even at high loadings of greater than 500 g/L-resin. Significant reductions in process-related impurities, including host cell protein, DNA, and model virus, have also been observed in pilot-scale operations, as well as bench-scale challenge studies.

FIG. 1B shows the step yield for the AEX step for the pilot-scale lots of FIG. 1A, demonstrating the ability of the step to achieve high yields while providing significant impurity reduction.

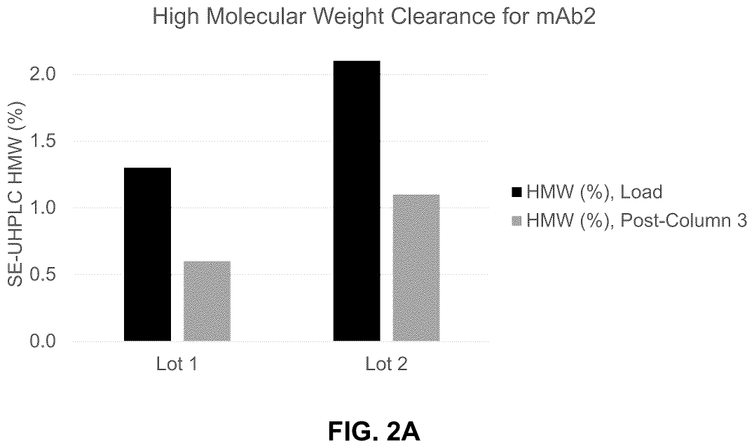

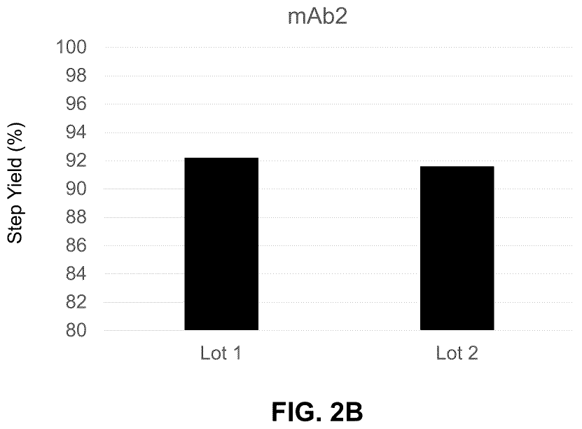

Similar high yields and significant impurity reduction was observed for mAb2. FIG. 2A shows high molecular weight clearance for mAb2 in two pilot-scale lots, and FIG. 2B shows the step yield for the AEX step for the pilot-scale lots of FIG. 2A.

### Example 2: Low pH Viral Inactivation Using Formic Acid

At certain manufacturing sites with equipment constraints, it is desirable to minimize the volume of the Protein A pool and subsequent pool titrations in order to ensure the volumes fit within vessel limitations, with a robust safety margin. As shown in Table 1, for an evaluation of the VI unit operation for mAb1 with a low pH incubation time of 60-90 minutes at 15-25° C., the use of 1M formic acid vs. 10% acetic acid as the acid titrant resulted in a lower acid volume required to achieve the pH for viral inactivation, a lower base volume required to neutralize the VI pool to pH 5.0, and a lower net volume expansion over the viral inactivation step. This is particularly advantageous when there is limited capability to reduce the Protein A elution pool volume, for instance, when operating at high column loadings.

TABLE 1
Impact of VI Titrant on mAb1 Pool Volume and Conductivity

| | | Test 2 | % Reduction |
|---|---|---|---|
| | | (PSL2, | (Formic Acid |
| | Test 1 | Lot 2 of | vs. Acetic |
| | (PSL1) | Example 1) | Acid) |
| Starting Material | mAb1 | mAb1 | — |
| | Protein | Protein | |
| | A pool | A pool | |
| Initial pH | 4.4 | 4.4 | — |
| Acid titrant | 10% Acetic | 1M Formic | — |
| | Acid | Acid | |
| Acid volume added (mL/L) | 107.3 | 36.1 | 66% |
| Acidified pH | 3.6 | ~3.6 | — |
| Base titrant | 2M Tris | 2M Tris | — |
| Base volume added (mL/L) | 67.6 | 24.6 | 64% |
| Final pH | 5.0 | 5.0 | — |
| Final Conductivity (mS/cm) | 6.4 | 4.5 | 30% |
| Net volume expansion over | 18% | 6% | 11% |
| viral inactivation | | | |

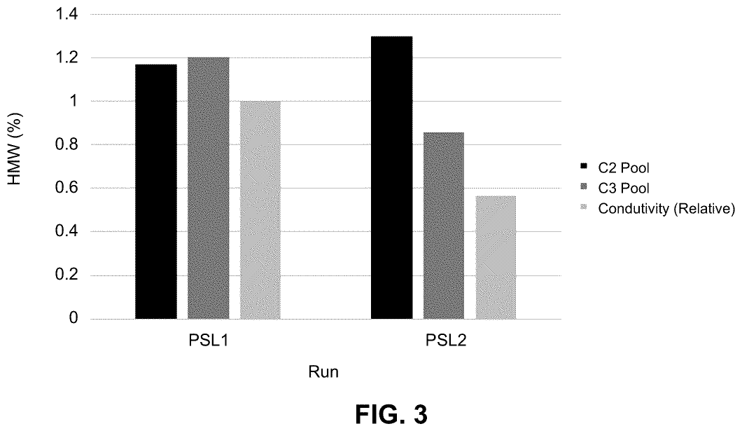

FIG. 3 demonstrates the benefits of lower load conductivity (resulting from the choice of the VI acidification titrant), as observed for a mAb1 AEX unit operation substantially similar to that described in Example 1. High molecular weight (HMW, detected by the SE-HPLC assay for mAb1) is often a critical quality attribute for mAbs such as mAb1, and HMW impurity levels are generally reduced by polishing chromatography steps such as AEX. FIG. 3 shows improved AEX step performance resulting from the use of a 1M formic acid VI titrant (PSL2), leading to a lower HMW percentage in the AEX pool vs. the use of a 10% acetic acid VI titrant (PSL1) under similar conditions, which exhibited minimal reduction in HMW species of mAb1. While the lower load conductivity reduced the step yield (93% for PSL2 versus 98% for PSL1) as a result of greater product retention on the resin, the AEX step yield when 1M formic acid was used as a VI titrant was still acceptable.

All documents, or portions of documents, cited in this application, including but not limited to patents, patent applications, articles, books, and treatises, are hereby expressly incorporated by reference. What is described in an embodiment of the disclosure can be combined with one or more other embodiments of the disclosure unless context clearly indicates otherwise.

The disclosed subject matter is not intended to be limited in scope by the specific embodiments described herein, which are instead intended as non-limiting illustrations of individual aspects of the disclosure. Functionally equivalent methods and components are within the scope of the disclosure. Indeed, various modifications of the disclosed subject matter, in addition to those shown and described herein, will be apparent to those skilled in the art from the foregoing description and accompanying drawing(s). Such modifications are intended to fall within the scope of the disclosed subject matter.

The descriptions of the various embodiments and/or examples of the disclosed subject matter have been presented for purposes of illustration, but are not intended to be exhaustive or limiting in any way. Many modifications and variations will be apparent to those of ordinary skill in the art without departing from the scope and spirit of the described embodiments. The terminology used herein was chosen to best explain the principles of the embodiments, the practical application, or technical improvement over technologies found in the marketplace, and/or to enable others of ordinary skill in the art to understand the disclosed subject matter.

What is claimed is:

1. A method for purifying a recombinant protein from a composition comprising the recombinant protein and at least one impurity, the method comprising:
loading the composition onto an anion exchange material comprising a primary amine ligand at a loading density of greater than about 100 g/L of anion exchange material, wherein:
the composition has a pH of about 7.0 to about 8.0 and a conductivity of less than about 10 mS/cm; and
the at least one impurity binds to the anion exchange material more strongly than the recombinant protein binds to the anion exchange material; and
collecting a purified composition comprising the recombinant protein.

2. The method of claim 1, wherein the anion exchange material comprises resin particles, wherein at least about 80% of the resin particles have a particle size of about 30 μm to about 60 μm.

3. The method of claim 1, wherein the anion exchange material comprises a polyamine ligand.

4. The method of claim 1, wherein the loading density is less than about 600 g/L of anion exchange material.

5. The method of claim 1, wherein the loading density is about 250 g/L-resin to about 600 g/L-resin.

6. The method of claim 1, wherein the composition has a conductivity of about 3 mS/cm to about 6 mS/cm.

7. The method of claim 1, wherein the method comprises using an equilibration buffer and/or a recovery buffer with the anion exchange material, wherein:
the pH of the equilibration buffer and/or the recovery buffer is about 7.0 to about 8.0;
and/or the conductivity of the equilibration buffer and/or the recovery buffer is less than about 10 mS/cm.

8. The method of claim 7, wherein the conductivity of the equilibration buffer and/or the recovery buffer is about 2 mS/cm to about 4 mS/cm.

9. The method of claim 1, further comprising performing a low pH viral inactivation unit operation one or more unit operations prior to the loading.

10. The method of claim 9, wherein the low pH viral inactivation unit operation employs an acid titrant comprising formic acid.

11. The method of claim 1, further comprising performing one or more additional chromatography unit operations.

12. The method of claim 11, wherein the one or more additional chromatography unit operations comprises an affinity chromatography unit operation performed prior to the loading.

13. The method of claim 12, wherein the affinity chromatography unit operation is selected from protein A chromatography, protein G chromatography, protein L chromatography, and CH1 domain chromatography.

14. The method of claim 11, wherein the one or more additional chromatography unit operations comprises an additional polishing chromatography unit operation.

15. The method of claim 14, wherein the additional polishing chromatography unit operation is selected from cation exchange chromatography, hydrophobic interaction chromatography, and mixed mode chromatography.

16. The method of claim 1, further comprising performing a viral filtration unit operation and/or a ultrafiltration/diafiltration (UF/DF) unit operation after the loading.

17. The method of claim 1, wherein:
less than about 2.5% w/w of the recombinant protein in the purified composition is high molecular weight species of the recombinant protein; and/or
the purified composition comprises at least about 85% w/w of the recombinant protein in the composition prior to the loading.

18. The method of claim 1, wherein the recombinant protein is an antigen-binding protein.

19. The method of claim 1, wherein the recombinant protein is an antibody.

20. The method of claim 1, wherein the at least one impurity is selected from host cell proteins, nucleic acids, high molecular weight species of the recombinant protein, fragments of the recombinant protein, cell culture media components, and viral contaminants.
